SEQUENCE LISTING (14 sequences)

SEQ ID NO: 1 - DNA, 1425 bp, Artificial Sequence
  Chemically synthesized, DMAb-V2L2 HC
atggactggacctggagaatcctgttcctggtggcagcagcaaccggaacacacgcagaggtgcagctgctggagagcggcggcggcctggtgcagcctggcggcagcctgaggctgtcctgcgcagcatctggcttcacctttagctcctatgcaatgaactgggtgcgccaggcaccaggcaagggactggagtgggtgtctgccatcacaatgagcggcatcaccgcctactatacagacgatgtgaagggcaggtttaccatcagcagagacaactccaagaatacactgtacctgcagatgaatagcctgagagccgaggataccgccgtgtactattgcgccaaggaggagttcctgcccggcacacactactattacggaatggacgtgtggggacagggaaccacagtgaccgtgtctagcgcctccacaaagggacctagcgtgttcccactggcaccctcctctaagtccacctctggcggcacagccgccctgggctgtctggtgaaggattatttcccagagcccgtgaccgtgtcttggaacagcggcgccctgacctctggagtgcacacatttccagccgtgctgcagagctccggcctgtatagcctgtctagcgtggtgaccgtgccctcctctagcctgggcacccagacatacatctgcaacgtgaatcacaagccatctaatacaaaggtggacaagaaggtggagcccaagagctgtgataagacccacacatgccctccctgtcctgcaccagagctgctgggcggcccatccgtgttcctgtttccacccaagcctaaggacaccctgatgatctcccggaccccagaggtgacatgcgtggtggtggacgtgtctcacgaggaccccgaggtgaagttcaactggtacgtggatggcgtggaggtgcacaatgccaagaccaagccacgggaggagcagtataacagcacctaccgcgtggtgtccgtgctgacagtgctgcaccaggactggctgaacggcaaggagtacaagtgcaaggtgagcaataaggccctgcccgcccctatcgagaagaccatctccaaggccaagggccagcctagggagccacaggtgtatacactgcctccaagcagagacgagctgaccaagaaccaggtgtccctgacatgtctggtgaagggcttctacccttccgatatcgccgtggagtgggagtctaatggccagccagagaacaattataagaccacaccccctgtgctggactccgatggctctttctttctgtactctaagctgaccgtggataagagccgctggcagcagggcaacgtgtttagctgttccgtgatgcacgaggccctgcacaatcactacacacagaagtctctgagcctgtcccctggcaagtgataa

SEQ ID NO: 2 - protein, 473 aa, Artificial Sequence
  Chemically synthesized, DMAb-V2L2 HC
MDWTWRILFLVAAATGTHAEVQLLESGGGLVQPGGSLRLSCAASGFTFSSYAMNWVRQAPGKGLEWVSAITMSGITAYYTDDVKGRFTISRDNSKNTLYLQMNSLRAEDTAVYYCAKEEFLPGTHYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 708 bp, Artificial Sequence
  Chemically synthesized, DMAb-V2L2 LC
atggtgctgcagacacaggtgttcatcagcctgctgctgtggatctccggagcatacggagcaatccagatgacccagtccccaagctccctgagcgcctccgtgggcgacagggtgaccatcacatgcagagcctctcagggcatccggaacgatctgggctggtaccagcagaagccaggcaaggcccccaagctgctgatctattctgccagcaccctgcagtctggagtgcccagccggttctccggctctggcagcggaacagactttaccctgacaatctctagcctgcagcctgaggacttcgccacctactattgcctgcaggattacaattatccatggacctttggccagggcacaaaggtggagatcaagcgcacagtggccgcccccagcgtgttcatctttccccctagcgacgagcagctgaagtccggcaccgcctctgtggtgtgcctgctgaacaatttctaccctagggaggccaaggtgcagtggaaggtggataacgccctgcagagcggcaattcccaggagtctgtgaccgagcaggacagcaaggattccacatattccctgtctaacaccctgacactgagcaaggccgattacgagaagcacaaggtgtatgcatgcgaggtgacccaccagggactgtcctctcccgtgacaaagtcctttaataggggcgagtgttgataa

SEQ ID NO: 4 - protein, 234 aa, Artificial Sequence
  Chemically synthesized, DMAb-V2L2 LC
MVLQTQVFISLLLWISGAYGAIQMTQSPSSLSASVGDRVTITCRASQGIRNDLGWYQQKPGKAPKLLIYSASTLQSGVPSRFSGSGSGTDFTLTISSLQPEDFATYYCLQDYNYPWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 5 - DNA, 425 bp, Artificial Sequence
  Chemically synthesized, DMAb-11-HC
atggactggacctggagaatcctgttcctggtggcagcagcaaccggaacacacgcagaggtgcagctggtggagagcggcggcggcctgatccagccaggcggcagcctgaggctgtcctgcgcagcatctggatttgccgtgaggagcaactacctgtcctgggtgagacaggcaccaggcaagggactggagtgggtgtctctgatctacagcggcggcctgaccgcatatgcagacagcgtggagggcaggttcaccatctccagagataactctaagaatacactgtatctgcagatgaattccctgcgggtggaggacaccgccctgtactattgcgcccgcgtggccagctccgccggcacattctactatggcatggacgtgtggggccagggcaccacagtgaccgtgtctagcgc

SEQ ID NO: 6 - protein, 141 aa, Artificial Sequence
  Chemically synthesized, DMAb-11-HC
MDWTWRILFLVAAATGTHAEVQLVESGGGLIQPGGSLRLSCAASGFAVRSNYLSWVRQAPGKGLEWVSLIYSGGLTAYADSVEGRFTISRDNSKNTLYLQMNSLRVEDTALYYCARVASSAGTFYYGMDVWGQGTTVTVSS

SEQ ID NO: 7 - DNA, 399 bp, Artificial Sequence
  Chemically synthesized, DMAb11-LC
atggtgctgcagacccaggtgtttatctctctgctgctgtggatcagcggcgcctacggcgatatcgtgatgacccagtcccctcgctccctgtctgtgacacctggcgagccagccagcatctcctgtcggtcctctcagtctctgctgcaccgcaacggctacaattatctggactggtacctgcagaagcccggccagtcccctcagctgctgatctatctgggcagcaacagggcatccggagtgccagaccgcttctctggcagcggctccggaaccgacttcaccctgaagatcagcagggtggaggccgaggatgtgggcgtgtactattgcatgcaggccctgcagaccccctcctggacattcggccagggcaccaaggtggagatcaag

SEQ ID NO: 8 - protein, 133 aa, Artificial Sequence
  Chemically synthesized, DMAb11-LC
MVLQTQVFISLLLWISGAYGDIVMTQSPRSLSVTPGEPASISCRSSQSLLHRNGYNYLDWYLQKPGQSPQLLIYLGSNRASGVPDRFSGSGSGTDFTLKISRVEAEDVGVYYCMQALQTPSWTFGQGTKVEIK

SEQ ID NO: 9 - DNA, 2241 bp, Artificial Sequence
  Chemically synthesized, Flu B
atggactggacttggaggattctgtttctggtggccgccgcaactggcactcatgccgaggtgcagctggtggaatcagggggaggactggtgaagcctggcggatcactgcgactgagctgcgcagcttccggactgaccttcctgaacgcttggatgagctgggtgcgacaggcaccagggaaaggcctggaatgggtcgggcgcatcaagagcaatacagacggcggaaccacagattacgcagcccccgtgaaaggcaggttcaccatttctcgggacgatagtaagaacacactgtatctgcagatgagctccctgaaaaccgaggacacagccgtgtactattgcactaccgatggcccctacagcgacgatttccgctccggatatgctgcacggtaccgctattttgggatggacgtgtggggacaggggacaactgtcacagtgtctagtgcatctactaagggacctagcgtgttcccactggccccctcaagcaaatcaactagcggagggaccgccgctctgggatgtctggtgaaggattacttccccgagcctgtcaccgtgagctggaactccggggccctgacctccggagtgcacacatttcctgctgtcctgcagtcctctgggctgtactctctgagttcagtggtcacagtgccaagctcctctctgggcactcagacctatatctgcaacgtgaatcacaaacctagcaatactaaggtcgacaagaaagtggaaccaaaaagctgtgataagacacatacttgccctccctgtccagctccagagctgctgggcggaccatccgtgttcctgtttccacccaagcccaaagacaccctgatgatttcccggacaccagaagtgacttgcgtggtcgtggacgtgagccacgaggaccccgaagtgaagttcaactggtacgtggatggcgtcgaggtgcataatgccaagacaaaacccagggaggaacagtacaactcaacttatagagtcgtgagcgtcctgaccgtgctgcaccaggactggctgaacggcaaggagtataagtgcaaagtgagcaacaaggccctgcctgctccaatcgagaagactattagcaaggctaaaggacagcctcgggaaccacaggtgtacaccctgcctccatcccgcgacgagctgaccaaaaaccaggtgtctctgacatgtctggtcaagggcttctatccctctgatatcgccgtggagtgggaaagtaatggacagcctgaaaacaattacaagaccacaccccctgtgctggactctgatggcagtttctttctgtatagtaaactgaccgtggacaagtcaagatggcagcagggaaacgtgttttcctgctctgtcatgcatgaggccctgcacaatcattacacccagaagagtctgtcactgagcccaggaaaacgagggaggaagaggagatccggctctggagccacaaacttctccctgctgaagcaggctggagacgtggaggaaaatcccgggcctatggtgctgcagacccaggtctttatctccctgctgctgtggatttctggcgcttacggagatatccagatgacacagtctcccagttcagtcagtgcatcagtgggcgaccgcgtcaccatcacatgtcgagcatcacaggatattagcacctggctggcctggtaccagcagaagcccggaaaagctcctaagctgctgatctatgcagccagctccctgcagtccggagtgccctctaggttcagcgggtccggctctggaacagactttactctgaccatttctagtctgcagcctgaggatttcgcaacttactattgccagcaggccaacagcttcccacccacttttgggcagggcaccaaactggaaatcaagactgtggctgcacctagcgtcttcatttttcctccatccgacgagcagctgaagagtggcaccgcctcagtggtgtgcctgctgaacaacttctacccaagagaagcaaaagtgcagtggaaggtcgataacgccctgcagtcaggcaatagccaggagtccgtgacagaacaggactctaaggatagtacttatagtctgtcaaatacactgactctgagcaaagctgactacgagaagcataaagtgtatgcatgcgaggtcactcaccagggactgtcttcacccgtcaccaaatctttcaatagaggagaatgctgataa

SEQ ID NO: 10 - protein, 745 aa, Artificial Sequence
  Chemically synthesized, Flu B
MDWTWRILFLVAAATGTHAEVQLVESGGGLVKPGGSLRLSCAASGLTFLNAWMSWVRQAPGKGLEWVGRIKSNTDGGTTDYAAPVKGRFTISRDDSKNTLYLQMSSLKTEDTAVYYCTTDGPYSDDFRSGYAARYRYFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSSVSASVGDRVTITCRASQDISTWLAWYQQKPGKAPKLLIYAASSLQSGVPSRFSGSGSGTDFTLTISSLQPEDFATYYCQQANSFPPTFGQGTKLEIKTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 11 - DNA, 1458 bp, Artificial Sequence
  Chemically synthesized, PGT128_HC
atggactggacctggagaatcctgttcctggtggcagcagcaacaggaacccacgcacagccacagctgcaggagtccggacccaccctggtggaggcctccgagacactgtctctgacctgcgccgtgagcggcgattccacagcagcctgtaactccttctggggatgggtgcgccagccccctggcaagggcctggagtgggtgggctctctgagccactgcgccagctactggaacaggggctggacctatcacaatccctctctgaagagcagactgaccctggccctggacacacctaagaacctggtgttcctgaagctgaatagcgtgaccgccgccgatacagccacctactattgtgccaggtttggcggcgaggtgctgagatacacagactggccaaagccagcatgggtggatctgtggggaaggggcacactggtgaccgtgagctccgcctccaccaagggaccaagcgtgttcccactggcaccttctagcaagtccacatctggcggcaccgccgccctgggatgcctggtgaaggactacttccctgagccagtgacagtgtcctggaactctggcgccctgacctctggcgtgcacacatttcccgccgtgctgcagtcctctggcctgtacagcctgagctccgtggtgaccgtgccttctagctccctgggcacacagacctatatctgcaacgtgaatcacaagcctagcaatacaaaggtggacaagaaggtggagccaaagtcctgtgataagacacacacctgcccaccctgtccagcacctgagctgctgggcggcccttccgtgttcctgtttcctccaaagccaaaggacaccctgatgatctcccggacacctgaggtgacctgcgtggtggtggacgtgtctcacgaggaccccgaggtgaagtttaactggtacgtggatggcgtggaggtgcacaatgccaagaccaagcccagggaggagcagtacaactctacctatagagtggtgagcgtgctgacagtgctgcaccaggattggctgaacggcaaggagtataagtgcaaggtgagcaataaggccctgccagcccccatcgagaagaccatctccaaggcaaagggacagccacgggagccacaggtgtacacactgcccccttcccgcgacgagctgaccaagaaccaggtgtctctgacatgtctggtgaagggcttctatccaagcgatatcgccgtggagtgggagtccaatggccagcccgagaacaattacaagaccacaccacccgtgctggacagcgatggctccttctttctgtattccaagctgaccgtggacaagtctaggtggcagcagggcaacgtgtttagctgctccgtgatgcacgaggccctgcacaatcactacacccagaagtctctgagcctgtcccctggcaagtgataa

SEQ ID NO: 12 - protein, 484 aa, Artificial Sequence
  Chemically synthesized, PGT128_HC
MDWTWRILFLVAAATGTHAQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 699 bp, Artificial Sequence
  Chemically synthesized, PGT128-LC
atggcctggacccctctgttcctgttcctgctgacatgctgtcctggcggctccaactctcagagcgccctgacccagcctccatccgcctctggcagccctggacagagcatcacaatctcctgtacaggcaccagcaacaatttcgtgagctggtaccagcagcacgcaggcaaggcaccaaagctggtcatctacgacgtgaacaagcggccttccggcgtgccagatcgcttctccggctctaagagcggcaatacagcctctctgaccgtgagcggcctgcagaccgacgatgaggccgtgtactattgcggcagcctggtgggcaactgggacgtgatcttcggcggcggaacaaagctgaccgtgctgggacagccaaaggcagcaccttccgtgaccctgtttcccccttctagcgaggagctgcaggccaataaggccaccctggtgtgcctgatcagcgacttctaccctggagcagtgacagtggcatggaaggccgattcctctccagtgaaggccggcgtggagaccacaaccccctctaagcagagcaacaataagtacgccgccagctcctatctgtctctgaccccagagcagtggaagagccacaagtcctattcttgccaggtgacacacgagggctctacagtggagaagaccgtggcccccacagagtgtagctgataa

SEQ ID NO: 14 - protein, 231 aa, Artificial Sequence
  Chemically synthesized, PGT128-LC
MAWTPLFLFLLTCCPGGSNSQSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

(19) United States
(12) Patent Application Publication
(10) Pub. No.: US 2023/0063151 A1
(43) Pub. Date: Mar. 2, 2023

(54) CD40-L BLOCKADE TO ENHANCE SYNTHETIC ANTIBODY THERAPY

(71) Applicant: The Wistar Institute of Anatomy and Biology, Philadelphia, PA (US)

(72) Inventors: David Weiner, Merion, PA (US); Ami Patel, Philadelphia, PA (US); Alfredo Perales-Puchalt, Philadelphia, PA (US); Megan C. Wise, Glenside, PA (US); Sarah T.C. Elliott, Pullman, WA (US)

(21) Appl. No.: 17/759,649

(22) PCT Filed: Jan. 29, 2021
(86) PCT No.: PCT/US21/15626

§ 371 Date: Jul. 28, 2022

Related U.S. Application Data
(60) Provisional application No. 62/967,486, filed on Jan. 29, 2020.

(51) Int. Cl. A61K 39/395; A61P 37/02; C07K 16/10; C07K 16/12; C07K 16/28

(52) U.S. Cl. CPC A61K 39/3955; A61P 37/02; C07K 16/1018; C07K 16/1045; C07K 16/1214; C07K 16/2875; A61K 2039/507

(57) ABSTRACT

Disclosed herein are combinations of inhibitors of B cell maturation and recombinant nucleic acid molecules encoding synthethic antibodies, and their use for extending the duration of circulating synthetic antibodies and for treating diseases and disorders.

### CROSS REFERENCE TO RELATED APPLICATIONS

This application claims priority to U.S. Provisional Application No. 62/967,486, filed Jan. 29, 2020 which is hereby incorporated by reference herein in its entirety.

### BACKGROUND

Both plasmid DNA and mRNA nucleic acid gene-encoded platforms are emerging as alternative approaches for in vivo delivery of monoclonal antibody (mAb) biologics. The synthetic DNA-encoded monoclonal antibody (DMAb) platform for in vivo antibody delivery, reporting expression and efficacy in infectious disease models was recently described (Elliott, S. T. C. et al., 2017, NPJ Vaccines, 2(1): 1-9; Patel A. et al., 2017, Nat. Commun., 8(1):1-11; Patel, A. et al., 2018, Cell Rep., 25(7):1982-1993; Muthumani, K. et al., 2016, J. Infect. Dis., 214(3):369-378; Wise, M. C. et al., 2019, J. Clin. Invest., 130(2))(1-5). DMAb has also been administered as alternatives to recombinant antibody biologics for lowering cholesterol (Khoshnejad, M. et al., 2019, Mol. Ther., 27(1):188-199), cancer (Muthumani, K. et al., 2017, Cancer Immunol. Immunother., 66(12):1577-1588; Perales-Puchalt, A. et al., 2019, JCI Insight, 4(8); Perales-Puchalt, A. et al., 2019, Oncatarget, 10(1):13-16; Duperret, E. K. et al., 2018, Cancer Res) and as a new strategy for delivery of bispecific T cell engagers (Perales-Puchalt, A. et al., 2019, JCI Insight, 4(8)). Like recombinant biologics, the development of anti-drug antibodies (ADA) against antibody complementarity determining regions (CDR) can dramatically impact biologic efficacy, potentially lowering potency, longevity in circulation, and impairing re-administration. The immunogenicity of chimeric antibodies derived from heterologous species, such as a mouse Fab on a human Fc, or even some fully human antibodies can dramatically shorten the circulating half-life of mAb biologics and gene-delivered antibodies. As CDR sequences are intrinsic to antibody functionality, alternative strategies that can reduce potential immunogenicity of these important biologics would be very beneficial. It was previously shown that T cell depletion, more specifically CD4+ T cell depletion, resulted in prolonged expression of DMAbs against Pseudomonas aeruginosa and other infectious diseases (Patel A. et al., 2017, Nat. Commun., 8(1):1-11; Patel, A. et al., 2018, Cell Rep., 25(7):1982-1993; Wise, M. C. et al., 2019, J. Clin. Invest., 130(2)). The anti-DMAb immune responses was determined to be specifically MHC Class II dependent (Patel, A. et al., 2018, Cell Rep., 25(7):1982-1993). As with recombinant antibodies, immune “self” vs “non-self” recognition plays an important role in development of ADA against gene-encoded antibodies such as DMAbs.

Thus, there is a need in the art for improved, cost-effective compositions and methods enhance immunogenicity of synthetic or recombinant antibodies. The current invention satisfies this unmet need.

### BRIEF SUMMARY OF THE INVENTION

In one embodiment, the invention relates to a composition comprising an inhibitor of B cell maturation or function and further comprising one or more nucleic acid molecules encoding one or more synthetic antibodies or fragments thereof. In one embodiment, the inhibitor of B cell maturation or function is an inhibitor of CD40L, CD20, CD22, VLA-4, BAFF or APRIL. In one embodiment, the inhibitor of B cell maturation or function is intravenous gamma globuli, interferon-β, DC2219, MR1, rituximab, Ocrelizumab, Epratuzumab, Atacicept, natalizumab or Belimumab.

In one embodiment, the nucleic acid molecule encodes a DNA encoded monoclonal antibody (DMAb), an ScFv DMAb, a DNA encoded bispecific T cell engager, a bispecific antibody, a chimeric antibody, or a functional antibody fragment.

In one embodiment, one or more nucleic acid molecules are engineered to be in an expression vector.

In one embodiment, the invention further comprises a checkpoint inhibitor, or nucleic acid molecule encoding the same. In one embodiment, the invention further comprises a pharmaceutically acceptable excipient.

In one embodiment, the invention relates to a method of treating a disease in a subject, the method comprising administering to the subject a composition comprising an inhibitor of B cell maturation or function and further comprising one or more nucleic acid molecules encoding one or more synthetic antibodies or fragments thereof. In one embodiment, the disease is a bacterial infection, a viral infection, a fungal infection, a disease or disorder associated with a parasite, or cancer.

In one embodiment, the invention relates to a method of extending the duration of circulation of a synthetic antibody, the method comprising administering to a subject in need thereof: a) an inhibitor of B cell maturation or function, and b) a composition comprising one or more nucleic acid molecule encoding a synthetic antibody. In one embodiment, the inhibitor of B cell maturation or function is an inhibitor of CD40L, CD20, CD22, VLA-4, BAFF or APRIL. In one embodiment, the inhibitor of B cell maturation or function is intravenous gamma globuli, interferon-β, DC2219, MR1, rituximab, Ocrelizumab, Epratuzumab, Atacicept, natalizumab or Belimumab. In one embodiment, the nucleic acid molecule encodes a DNA encoded monoclonal antibody (DMAb), an ScFv DMAb, a DNA encoded bispecific T cell engager, a bispecific antibody, a chimeric antibody, or a functional antibody fragment. In one embodiment, the one or more nucleic acid molecules are engineered to be in an expression vector. In one embodiment, administering the composition comprises an electroporating step. In one embodiment, the method further comprises a step of administering to the subject a composition comprising an antigen.

In one embodiment, the invention relates to a method of treating or preventing a disease or disorder, the method comprising administering to a subject in need thereof: a) an inhibitor of B cell maturation or function, and b) a composition comprising one or more nucleic acid molecule encoding a synthetic antibody. In one embodiment, the inhibitor of B cell maturation or function is an inhibitor of CD40L, CD20, CD22, VLA-4, BAFF or APRIL. In one embodiment, the inhibitor of B cell maturation or function is intravenous gamma globuli, interferon-β, DC2219, MR1, rituximab, Ocrelizumab, Epratuzumab, Atacicept, natalizumab or Belimumab. In one embodiment, the nucleic acid molecule encodes a DNA encoded monoclonal antibody (DMAb), an ScFv DMAb, a DNA encoded bispecific T cell engager, a bispecific antibody, a chimeric antibody, or a functional antibody fragment. In one embodiment, the one or more nucleic acid molecules are engineered to be in an expression vector. In one embodiment, the disease is a bacterial infection, a viral infection, a fungal infection, a disease or disorder associated with a parasite, or cancer.

### BRIEF DESCRIPTION OF THE DRAWINGS

FIG. 1A through FIG. 1C: Schematic of DMAbs. (FIG. 1A) DMAb structure for cloning in one (top) or two (bottom) plamids. P2A: porcine teschovirus-1 2A sequence. (FIG. 1B) Representation of DMAb cloned into pVAX1 plasmid. (FIG. 1C) Resulting antibody generic structure from DMAb. hIGELS: human IgE leader sequence. LC: light chain, HC: heavy chain.

FIG. 2A through FIG. 2D: Development of T cell responses following delivery of human DMAbs in mice.

FIG. 3A through FIG. 3C: anti-DMAb immune responses when administered a a locally delivered glucorticoid.

FIG. 4: Co-delivery of rapamycin successfully increased the DMAb PK

FIG. 5: Blocking T-cell co-stimulation with CTLA4-Ig.

FIG. 6A through FIG. 6D: CD40L blockade results in increased half-life of DMAbs. (FIG. 6A) Schematic of experiment. (FIG. 6B) V2L2 DMAb concentration in C57B1/6 sera with or without CD40L blockade. (FIG. 6C) Mouse anti-V2L2 DMAb IgG at days 7, 56 and 259 in C57B1/6 mice with or without CD40L blockade. (FIG. 6D) V2L2 DMAb concentration and anti-V2L2 DMAb IgG at day 194 in individual mice.

FIG. 7A through FIG. 7F: Validation of CD40L blockade in HIV and Flu DMAbs. (FIG. 7A) PGT128 DMAb concentration in C57Bl/6 sera with or without CD40L blockade (n=5 mice per group). (FIG. 7B) FluA DMAb concentration in C57Bl/6 sera with or without CD40L blockade (n=5 mice per group). (FIG. 7C) Mouse anti-FluA DMAb IgG at days 0, 6, 17, 24 and 89 in Balb/c mice with CD40L blockade, T cell depletion or IgG control. (FIG. 7D) Mouse anti-FluA DMAb IgG at day 89 (1:25 serum dilution) with CD40L blockade, T cell depletion or IgG control. (FIG. 7E) FluA DMAb ability to bind to recombinant HA1 from Flu A H3 by ELISA using sera from day 184 of mice treated with FluA DMAb and CD40L blockade or isotype control or treated with pVax empty vector. (FIG. 7F) Endpoint titer of human FluA DMAb in sera from days 89 and 184 of mice treated with FluA DMAb and CD40L blockade or isotype control. One-way ANOVA, Two-way ANOVA. *p<0.05, ***p<0.001, ns: not significant.

FIG. 8A through FIG. 8D: Anti-DMAb antibodies result in DMAb clearance from mouse sera. (FIG. 8A) Schematic of experiment. Human IgG V2L2 was delivered via intramuscular injection with electroporation at Day 0 and sera were collected over a period of six months. (FIG. 8B) Mouse anti-V2L2 DMAb IgG over time after DMAb expression in C57Bl/6 or B cell deficient (Mu^{-/-}) mice. (FIG. 8C) V2L2 DMAb levels in C57Bl/6 or B cell deficient (muMt⁻) mouse sera.

FIG. 9A through FIG. 9C: Immune recovery after MR1 treatment. (FIG. 9A) Schematic of experiment: 3 groups of n=5 mice were treated with 500 ug of anti-CD40L antibody MR1 14, 7 or on the same day of receiving a Flu H3 vaccine. Mice were euthanized 14 days after vaccination and immune responses measured by (FIG. 9B) interferon gamma ELISPOT and (FIG. 9C) binding ELISA. One-way ANOVA. Two-way ANOVA. *p<0.05, **p<0.01, ns: not significant.

### DETAILED DESCRIPTION

The present invention relates to compositions and methods for inhibiting B cell maturation and function in combination with administration of synthetic antibodies, fragments thereof, variants thereof, or a combination thereof to increase the immunogenicity of the synthetic antibody. In some embodiments, the method of inhibiting B cell maturation and function is through transient blockade of CD40L.

In one aspect, the present invention relates to a composition that can be used to increase or enhance an immune response, i.e., create a more effective immune response, by administering an inhibitor of CD40L in combination with a synthetic antibody, fragment thereof, variant thereof, or a combination thereof. In one embodiment, the inhibitor of CD40L is an anti-CD40L antibody. In one embodiment, the inhibitor of CD40L is an anti-CD40L antibody MR1.

With respect to engineered synthetic DNA antibodies in the form of synthetic DNA plasmids, the present invention relates to compositions comprising an inhibitor of CD40L in combination with a recombinant nucleic acid sequence encoding an antibody, a fragment thereof, a variant thereof, or a combination thereof. The composition can be administered to a subject in need thereof to facilitate in vivo expression and formation of the synthetic antibody.

In some instances, the combination of CD40L inhibitor and synthetic antibody of the invention can be administered in combination with one or more additional antibodies targeting checkpoint molecules (e.g., anti-CTLA4 antibodies), to produce a synergistic effect; whereas, in other instances, the combination of CD40L inhibitor and synthetic antibody of the invention can be administered alone.

In some instances, the combination of CD40L inhibitor and synthetic antibody of the invention of the invention can be administered in combination with a desired composition comprising an antigen, such as TERT, to produce a synergistic effect; whereas, in other instances, the combination of CD40L inhibitor and synthetic antibody of the invention can be administered separately from the composition comprising the antigen.

The compositions provided herein can also include a pharmaceutically acceptable excipient.

The composition of the present invention can increase the immune response to the antigen of the vaccine in the subject by suppressing the B cell response while increasing the CD8⁺ T cell response, as compared to the administration of the synthetic antibody alone. This increased CD8⁺ T cell response has cytolytic activity and secretes the cytokine interferon-gamma (IFN-γ).

### 1. DEFINITIONS

Unless otherwise defined, all technical and scientific terms used herein have the same meaning as commonly understood by one of ordinary skill in the art. In case of conflict, the present document, including definitions, will control. Exemplary methods and materials are described herein, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Antibody” may mean an antibody of classes IgG, IgM, IgA, IgD or IgE, or fragments, fragments or derivatives thereof, including Fab, F(ab′)2, Fd, and single chain antibodies, and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

“Antigen” refers to proteins that have the ability to generate an immune response in a host. An antigen may be recognized and bound by an antibody. An antigen may originate from within the body or from the external environment.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p 877-883.

“Antibody fragment” or “fragment of an antibody” as used interchangeably herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Adjuvant” as used herein means any molecule added to the vaccine described herein to enhance the immunogenicity of the antigen.

“Checkpoint inhibitor” as used herein means inhibitors or molecules that block immune checkpoints as commonly understood in the field of cancer immunotherapy. More commonly the checkpoint inhibitors are antibodies that block these immune checkpoints.

“Coding sequence” or “encoding nucleic acid” as used herein may refer to the nucleic acid (RNA or DNA molecule) that comprise a nucleotide sequence which encodes an antibody as set forth herein. The coding sequence may also comprise a DNA sequence which encodes an RNA sequence. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides.

“Complement” or “complementary” as used herein may mean a nucleic acid may have Watson-Crick (e.g., A-T/U and C-G) or Hoogsteen base pairing between nucleotides or nucleotide analogs of nucleic acid molecules. “Constant current” as used herein to define a current that is received or experienced by a tissue, or cells defining said tissue, over the duration of an electrical pulse delivered to same tissue. The electrical pulse is delivered from the electroporation devices described herein. This current remains at a constant amperage in said tissue over the life of an electrical pulse because the electroporation device provided herein has a feedback element, preferably having instantaneous feedback. The feedback element can measure the resistance of the tissue (or cells) throughout the duration of the pulse and cause the electroporation device to alter its electrical energy output (e.g., increase voltage) so current in same tissue remains constant throughout the electrical pulse (on the order of microseconds), and from pulse to pulse. In some embodiments, the feedback element comprises a controller.

“Current feedback” or “feedback” as used herein may be used interchangeably and may mean the active response of the provided electroporation devices, which comprises measuring the current in tissue between electrodes and altering the energy output delivered by the EP device accordingly in order to maintain the current at a constant level. This constant level is preset by a user prior to initiation of a pulse sequence or electrical treatment. The feedback may be accomplished by the electroporation component, e.g., controller, of the electroporation device, as the electrical circuit therein is able to continuously monitor the current in tissue between electrodes and compare that monitored current (or current within tissue) to a preset current and continuously make energy-output adjustments to maintain the monitored current at preset levels. The feedback loop may be instantaneous as it is an analog closed-loop feedback.

“Decentralized current” as used herein may mean the pattern of electrical currents delivered from the various needle electrode arrays of the electroporation devices described herein, wherein the patterns minimize, or preferably eliminate, the occurrence of electroporation related heat stress on any area of tissue being electroporated.

“Electroporation,” “electro-permeabilization,” or “electro-kinetic enhancement” (“EP”) as used interchangeably herein may refer to the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Endogenous antibody” as used herein may refer to an antibody that is generated in a subject that is administered an effective dose of an antigen for induction of a humoral immune response.

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

“Fragment” may mean a polypeptide fragment of an antibody that is function, i.e., can bind to desired target and have the same intended effect as a full length antibody. A fragment of an antibody may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antibody, excluding any heterologous signal peptide added. The fragment may comprise a fragment of a polypeptide that is 95% or more, 96% or more, 97% or more, 98% or more or 99% or more identical to the antibody and additionally comprise an N terminal methionine or heterologous signal peptide which is not included when calculating percent identity. Fragments may further comprise an N terminal methionine and/or a signal peptide such as an immunoglobulin signal peptide, for example an IgE or IgG signal peptide. The N terminal methionine and/or signal peptide may be linked to a fragment of an antibody.

A fragment of a nucleic acid sequence that encodes an antibody may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antibody and additionally optionally comprise sequence encoding an N terminal methionine or heterologous signal peptide which is not included when calculating percent identity. Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

“Genetic construct” as used herein refers to the DNA or RNA molecules that comprise a nucleotide sequence which encodes a protein, such as an antibody. The genetic construct may also refer to a DNA molecule which transcribes an RNA. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Impedance” as used herein may be used when discussing the feedback mechanism and can be converted to a current value according to Ohm's law, thus enabling comparisons with the preset current.

“Immune response” as used herein may mean the activation of a host's immune system, e.g., that of a mammal, in response to the introduction of one or more nucleic acids and/or peptides. The immune response can be in the form of a cellular or humoral response, or both.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter,

SV40 late promoter, SV40 early promoter, RSV-LTR promoter, CMV IE promoter, SV40 early promoter or SV 40 late promoter and the CMV IE promoter.

“Signal peptide” and “leader sequence” are used interchangeably herein and refer to an amino acid sequence that can be linked at the amino terminus of a protein set forth herein. Signal peptides/leader sequences typically direct localization of a protein.

Signal peptides/leader sequences used herein may facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein. “Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. The subject or patient may be undergoing other forms of treatment.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

“Synthetic antibody” as used herein refers to an antibody that is encoded by the recombinant nucleic acid sequence described herein and is generated in a subject.

“Treatment” or “treating,” as used herein can mean protecting of a subject from a disease through means of preventing, suppressing, repressing, or completely eliminating the disease. Preventing the disease involves administering a vaccine of the present invention to a subject prior to onset of the disease. Suppressing the disease involves administering a vaccine of the present invention to a subject after induction of the disease but before its clinical appearance. Repressing the disease involves administering a vaccine of the present invention to a subject after clinical appearance of the disease.

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide, may indicate that the peptide or polypeptide differs in amino acid sequence by the insertion, deletion, or conservative substitution of amino acids, but retains at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. This applies regardless of the breadth of the range.

### 2. COMPOSITIONS

In one embodiment, the invention provides a composition comprising an inhibitor of B cell maturation and function for use in combination with one or more synthetic antibody. In one embodiment, the invention provides a composition comprising an inhibitor of B cell maturation and one or more synthetic antibody. In one embodiment, the synthetic antibodies of the invention can be produced in mammalian cells or for delivery in DNA or RNA vectors including bacterial, yeast, as well as viral vectors.

In one embodiment, the compositions comprising synthetic antibodies of the invention include a recombinant nucleic acid sequence encoding an antibody, a fragment thereof, a variant thereof, or a combination thereof. The composition, when administered to a subject in need thereof, can result in the generation of a synthetic antibody in the subject. The synthetic antibody can bind a target molecule (i.e., an antigen) present in the subject. Such binding can neutralize the antigen, block recognition of the antigen by another molecule, for example, a protein or nucleic acid, and elicit or induce an immune response to the antigen.

In one embodiment, the composition comprises a nucleotide sequence encoding a synthetic antibody. In one embodiment, the composition comprises a nucleic acid molecule comprising a first nucleotide sequence encoding a first synthetic antibody and a second nucleotide sequence encoding a second synthetic antibody. In one embodiment, the nucleic acid molecule comprises a nucleotide sequence encoding a cleavage domain.

The composition of the invention can treat, prevent, and/or protect against any disease, disorder, or condition associated with the antigen target of the synthetic antibody. In certain embodiments, the composition can treat, prevent, and/or protect against cancer. In one embodiment, the composition of the invention is provided in combination with at least one other agent, such as an antigen.

In one embodiment, a combination of inhibitor of B cell maturation and synthetic antibody, or recombinant nucleic acid molecule encoding the same, can be a single formulation or can be separate formulations and administered in sequence (either inhibitor of B cell maturation first and then synthetic antibody, or recombinant nucleic acid molecule encoding the same, or synthetic antibody, or recombinant nucleic acid molecule encoding the same first and then inhibitor of B cell maturation). The composition can extending the duration of circulation of the synthetic antibody in the subject thereby increasing the overall immune response to the targeted antigen in a subject. The combination of inhibitor of B cell maturation and synthetic antibody, or recombinant nucleic acid molecule encoding the same, induces a greater antigen-specific immune response than a composition comprising the synthetic antibody, or recombinant nucleic acid molecule encoding the same alone. This more efficient immune response provides increased efficacy in the treatment and/or prevention of a disease, such as cancer.

The composition can result in the generation of the synthetic antibody in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, 84 hours, or 96 hours. The composition comprising an inhibitor of B cell maturation can be administered before or after administration of the synthetic antibody, or nucleic acid molecule encoding the same, to the subject. In some embodiments, the inhibitor of B cell maturation can be administered at least 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 48 hours, 72 hours, or more than 72 hours before or after administration of the synthetic antibody, or nucleic acid molecule encoding the same, to the subject.

In some embodiments, administration of the inhibitor of B cell maturation can result in the suppression of B cell maturation or function within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, 84 hours, or 96 hours. The suppression of B cell maturation or function can occur before or after administration of the synthetic antibody, or nucleic acid molecule encoding the same, to the subject.

The compositions of the present invention can have features required of effective compositions such as being safe so that the composition does not cause illness or death; being protective against illness; and providing ease of administration, few side effects, biological stability, and low cost per dose. The composition may accomplish some or all of these features by combining the antigen(s) with the checkpoint inhibitor(s), such as an anti-PD-1 antibody as discussed herein.

Inhibitors of B Cell Maturation

In one embodiment, the invention provides compositions comprising inhibitors of B cell maturation for use in combination with a synthetic antibody, or a recombinant nucleic acid sequence encoding a synthetic antibody. In one embodiment, the inhibitor of the invention decreases the amount of polypeptide, the amount of mRNA, the amount of activity, or a combination thereof of a protein which functions in B cell proliferation, maturation, antibody secretion, or a combination thereof.

It will be understood by one skilled in the art, based upon the disclosure provided herein, that a decrease in the level of polypeptide encompasses the decrease in the expression, including transcription, translation, or both. The skilled artisan will also appreciate, once armed with the teachings of the present invention, that a decrease in the level of polypeptide includes a decrease in the activity of the protein. Thus, decrease in the level or activity of a protein which functions in B cell proliferation, maturation, antibody secretion, or a combination thereof includes, but is not limited to, decreasing the amount of polypeptide of the protein which functions in B cell proliferation, maturation, antibody secretion, or a combination thereof, and decreasing transcription, translation, or both, of a nucleic acid encoding the protein which functions in B cell proliferation, maturation, antibody secretion, or a combination thereof; and it also includes decreasing any activity of the protein which functions in B cell proliferation, maturation, antibody secretion, or a combination thereof, as well.

In one embodiment, the invention provides a generic concept for inhibiting B cell maturation or function in combination with providing a synthetic antibody as a therapy. In one embodiment, the composition of the invention comprises an inhibitor of B cell maturation or function. In one embodiment, the inhibitor is selected from the group consisting of a small interfering RNA (siRNA), a microRNA, an antisense nucleic acid, a ribozyme, an expression vector encoding a transdominant negative mutant, an intracellular antibody, a peptide and a small molecule.

One skilled in the art will appreciate, based on the disclosure provided herein, that one way to decrease the mRNA and/or protein levels of a protein which functions in B cell proliferation, maturation, antibody secretion, or a combination thereof is by reducing or inhibiting expression of the nucleic acid encoding the protein which functions in B cell proliferation, maturation, antibody secretion, or a combination thereof. Thus, the level of a protein which functions in B cell proliferation, maturation, antibody secretion, or a combination thereof can also be decreased using a molecule or compound that inhibits or reduces gene expression such as, for example, siRNA, an antisense molecule or a ribozyme. However, the invention should not be limited to these examples.

In one embodiment, siRNA is used to decrease the level of a protein which functions in B cell proliferation, maturation, antibody secretion, or a combination thereof. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, Pa. (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing protein level using RNAi technology.

In other related aspects, the invention includes an isolated nucleic acid encoding an inhibitor, wherein an inhibitor such as an siRNA or antisense molecule, inhibits a protein which functions in B cell proliferation, maturation, antibody secretion, or a combination thereof, a derivative thereof, a regulator thereof, or a downstream effector, operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, N.Y.) and as described elsewhere herein. In another aspect of the invention, a protein which functions in B cell proliferation, maturation, antibody secretion, or a combination thereof or a regulator thereof, can be inhibited by way of inactivating and/or sequestering one or more of the protein, or a regulator thereof. As such, inhibiting the effects of a protein which functions in B cell proliferation, maturation, antibody secretion, or a combination thereof can be accomplished by using a transdominant negative mutant.

In another aspect, the invention includes a vector comprising an siRNA or antisense polynucleotide. Preferably, the siRNA or antisense polynucleotide is capable of inhibiting the expression of the protein which functions in B cell proliferation, maturation, antibody secretion, or a combination thereof. The incorporation of a desired polynucleotide into a vector and the choice of vectors is well-known in the art as described in, for example, Sambrook et al., supra.

The siRNA or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA or antisense polynucleotide, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

In one embodiment of the invention, an antisense nucleic acid sequence which is expressed by a plasmid vector is used to inhibit a protein which functions in B cell proliferation, maturation, antibody secretion, or a combination thereof. The antisense expressing vector is used to transfect a mammalian cell or the mammal itself, thereby causing reduced endogenous expression of the protein which functions in B cell proliferation, maturation, antibody secretion, or a combination thereof.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

Compositions and methods for the synthesis and expression of antisense nucleic acids are as described elsewhere herein.

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In one embodiment of the invention, a ribozyme is used to inhibit a protein which functions in B cell proliferation, maturation, antibody secretion, or a combination thereof. Ribozymes useful for inhibiting the expression of a target molecule may be designed by incorporating target sequences into the basic ribozyme structure which are complementary, for example, to the mRNA sequence of a protein which functions in B cell proliferation, maturation, antibody secretion, or a combination thereof of the present invention. Ribozymes may be synthesized using commercially available reagents (Applied Biosystems, Inc., Foster City, Calif.) or they may be genetically expressed from DNA encoding them.

When the inhibitor of the invention is a small molecule, a small molecule antagonist may be obtained using standard methods known to the skilled artisan. Such methods include chemical organic synthesis or biological means. Biological means include purification from a biological source, recombinant synthesis and in vitro translation systems, using methods well known in the art.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core—building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

As will be understood by one skilled in the art, any antibody that can inhibit a protein which functions in B cell proliferation, maturation, antibody secretion, or a combination thereof is useful in the present invention. Methods of making and using antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, bind to the specific antigens of interest, and they are able to bind the antigen present on Western blots, in solution in enzyme linked immunoassays, in fluorescence activated cells sorting (FACS) assays, in magnetic affinity cell sorting (MACS) assays, and in immunofluorescence microscopy of a cell transiently transfected with a nucleic acid encoding at least a portion of the antigenic protein, for example.

One skilled in the art would appreciate, based upon the disclosure provided herein, that the antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

The skilled artisan would appreciate, based upon the disclosure provided herein, that that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest. When the antibody used in the invention is humanized, the antibody may be generated as described in Queen, et al. (U.S. Pat. No. 6, 180,370), Wright et al., (supra) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as an epitope on an antigen of interest, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

The invention also includes functional equivalents of the antibodies described herein. Functional equivalents have binding characteristics comparable to those of the antibodies, and include, for example, hybridized and single chain antibodies, as well as fragments thereof. Methods of producing such functional equivalents are disclosed in PCT Application WO 93/21319 and PCT Application WO 89/09622.

Functional equivalents include polypeptides with amino acid sequences substantially the same as the amino acid sequence of the variable or hypervariable regions of the antibodies. “Substantially the same” amino acid sequence is defined herein as a sequence with at least 70%, preferably at least about 80%, more preferably at least about 90%, even more preferably at least about 95%, and most preferably at least 99% homology to another amino acid sequence (or any integer in between 70 and 99), as determined by the FASTA search method in accordance with Pearson and Lipman, 1988 Proc. Nat'l. Acad. Sci. USA 85: 2444-2448. Chimeric or other hybrid antibodies have constant regions derived substantially or exclusively from human antibody constant regions and variable regions derived substantially or exclusively from the sequence of the variable region of a monoclonal antibody from each stable hybridoma.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab′)₂ fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H₂L₂) formed of two dimers associated through at least one disulfide bridge.

In some embodiments, proteins which function in B cell proliferation, maturation, antibody secretion, or a combination thereof include, but are not limited to, CD40L inhibitors, CD20 inhibitors, CD22 inhibitors, BAFF inhibitors, and APRIL inhibitors.

In some embodiments, the inhibitor of B cell maturation and function is an antibody targeting CD40L, CD20, CD22, VLA-4, BAFF or APRIL. Antibodies targeting B cell expressed proteins include, but are not limited to, MR1, rituximab, Ocrelizumab, Epratuzumab, Atacicept, natalizumab and Belimumab.

Other inhibitors of B cell maturation and function include, but are not limited to, intravenous gamma globuli, interferon-β, and DC2219 (a recombinant immunotoxin).

Recombinant Nucleic Acid Sequence

As described above, the composition can comprise a recombinant nucleic acid sequence. The recombinant nucleic acid sequence can encode the synthetic antibody (e.g., DMAb, ScFv antibody fragment or DBiTE), a fragment thereof, a variant thereof, or a combination thereof. The antibody is described in more detail below.

The recombinant nucleic acid sequence can be a heterologous nucleic acid sequence. The recombinant nucleic acid sequence can include at least one heterologous nucleic acid sequence or one or more heterologous nucleic acid sequences.

The recombinant nucleic acid sequence can be an optimized nucleic acid sequence. Such optimization can increase or alter the immunogenicity of the antibody. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

The recombinant nucleic acid sequence can include one or more recombinant nucleic acid sequence constructs. The recombinant nucleic acid sequence construct can include one or more components, which are described in more detail below.

The recombinant nucleic acid sequence construct can include a heterologous nucleic acid sequence that encodes a heavy chain polypeptide, a fragment thereof, a variant thereof, or a combination thereof. The recombinant nucleic acid sequence construct can include a heterologous nucleic acid sequence that encodes a light chain polypeptide, a fragment thereof, a variant thereof, or a combination thereof. The recombinant nucleic acid sequence construct can also include a heterologous nucleic acid sequence that encodes a protease or peptidase cleavage site. The recombinant nucleic acid sequence construct can also include a heterologous nucleic acid sequence that encodes an internal ribosome entry site (IRES). An IRES may be either a viral IRES or an eukaryotic IRES. The recombinant nucleic acid sequence construct can include one or more leader sequences, in which each leader sequence encodes a signal peptide. The recombinant nucleic acid sequence construct can include one or more promoters, one or more introns, one or more transcription termination regions, one or more initiation codons, one or more termination or stop codons, and/or one or more polyadenylation signals. The recombinant nucleic acid sequence construct can also include one or more linker or tag sequences. The tag sequence can encode a hemagglutinin (HA) tag.

Heavy Chain Polypeptide

The recombinant nucleic acid sequence construct can include a heterologous nucleic acid encoding a heavy chain polypeptide, a fragment thereof, a variant thereof, or a combination thereof. The heavy chain polypeptide can include a variable heavy chain (VH) region and/or at least one constant heavy chain (CH) region. The at least one constant heavy chain region can include a constant heavy chain region 1 (CH1), a constant heavy chain region 2 (CH2), and a constant heavy chain region 3 (CH3), and/or a hinge region.

In some embodiments, the heavy chain polypeptide can include a VH region and a CH1 region. In other embodiments, the heavy chain polypeptide can include a VH region, a CH1 region, a hinge region, a CH2 region, and a CH3 region.

The heavy chain polypeptide can include a complementarity determining region (“CDR”) set. The CDR set can contain three hypervariable regions of the VH region. Proceeding from N-terminus of the heavy chain polypeptide, these CDRs are denoted “CDR1,” “CDR2,” and “CDR3,” respectively. CDR1, CDR2, and CDR3 of the heavy chain polypeptide can contribute to binding or recognition of the antigen.

Light Chain Polypeptide

The recombinant nucleic acid sequence construct can include a heterologous nucleic acid sequence encoding a light chain polypeptide, a fragment thereof, a variant thereof, or a combination thereof. The light chain polypeptide can include a variable light chain (VL) region and/or a constant light chain (CL) region.

The light chain polypeptide can include a complementarity determining region (“CDR”) set. The CDR set can contain three hypervariable regions of the VL region. Proceeding from N-terminus of the light chain polypeptide, these CDRs are denoted “CDR1,” “CDR2,” and “CDR3,” respectively. CDR1, CDR2, and CDR3 of the light chain polypeptide can contribute to binding or recognition of the antigen.

Protease Cleavage Site

The recombinant nucleic acid sequence construct can include the heterologous nucleic acid sequence encoding the protease cleavage site. The protease cleavage site can be recognized by a protease or peptidase. The protease can be an endopeptidase or endoprotease, for example, but not limited to, furin, elastase, HtrA, calpain, trypsin, chymotrypsin, trypsin, and pepsin. The protease can be furin. In other embodiments, the protease can be a serine protease, a threonine protease, cysteine protease, aspartate protease, metalloprotease, glutamic acid protease, or any protease that cleaves an internal peptide bond (i.e., does not cleave the N-terminal or C-terminal peptide bond).

The protease cleavage site can include one or more amino acid sequences that promote or increase the efficiency of cleavage. The one or more amino acid sequences can promote or increase the efficiency of forming or generating discrete polypeptides. The one or more amino acids sequences can include a 2A peptide sequence.

Linker Sequence

The recombinant nucleic acid sequence construct can include one or more linker sequences. The linker sequence can spatially separate or link the one or more components described herein. In other embodiments, the linker sequence can encode an amino acid sequence that spatially separates or links two or more polypeptides.

Promoter

The recombinant nucleic acid sequence construct can include one or more promoters. The one or more promoters may be any promoter that is capable of driving gene expression and regulating gene expression. Such a promoter is a cis-acting sequence element required for transcription via a DNA dependent RNA polymerase. Selection of the promoter used to direct gene expression depends on the particular application. The promoter may be positioned about the same distance from the transcription start in the recombinant nucleic acid sequence construct as it is from the transcription start site in its natural setting. However, variation in this distance may be accommodated without loss of promoter function.

The promoter may be operably linked to the heterologous nucleic acid sequence encoding the heavy chain polypeptide and/or light chain polypeptide. The promoter may be a promoter shown effective for expression in eukaryotic cells. The promoter operably linked to the coding sequence may be a CMV promoter, a promoter from simian virus 40 (SV40), such as SV40 early promoter and SV40 later promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, human polyhedrin, or human metalothionein.

The promoter can be a constitutive promoter or an inducible promoter, which initiates transcription only when the host cell is exposed to some particular external stimulus. In the case of a multicellular organism, the promoter can also be specific to a particular tissue or organ or stage of development. The promoter may also be a tissue specific promoter, such as a muscle or skin specific promoter, natural or synthetic. Examples of such promoters are described in US patent application publication no. US20040175727, the contents of which are incorporated herein in its entirety.

The promoter can be associated with an enhancer. The enhancer can be located upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

Transcription Termination Region

The recombinant nucleic acid sequence construct can include one or more transcription termination regions. The transcription termination region can be downstream of the coding sequence to provide for efficient termination. The transcription termination region can be obtained from the same gene as the promoter described above or can be obtained from one or more different genes.

Initiation Codon

The recombinant nucleic acid sequence construct can include one or more initiation codons. The initiation codon can be located upstream of the coding sequence. The initiation codon can be in frame with the coding sequence. The initiation codon can be associated with one or more signals required for efficient translation initiation, for example, but not limited to, a ribosome binding site.

Termination Codon

The recombinant nucleic acid sequence construct can include one or more termination or stop codons. The termination codon can be downstream of the coding sequence. The termination codon can be in frame with the coding sequence. The termination codon can be associated with one or more signals required for efficient translation termination.

Polyadenylation Signal

The recombinant nucleic acid sequence construct can include one or more polyadenylation signals. The polyadenylation signal can include one or more signals required for efficient polyadenylation of the transcript. The polyadenylation signal can be positioned downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, Calif.).

Leader Sequence

The recombinant nucleic acid sequence construct can include one or more leader sequences. The leader sequence can encode a signal peptide. The signal peptide can be an immunoglobulin (Ig) signal peptide, for example, but not limited to, an IgG signal peptide and a IgE signal peptide.

Expression from the Recombinant Nucleic Acid Sequence Construct

As described above, the recombinant nucleic acid sequence construct can include, amongst the one or more components, the heterologous nucleic acid sequence encoding the heavy chain polypeptide and/or the heterologous nucleic acid sequence encoding the light chain polypeptide. Accordingly, the recombinant nucleic acid sequence construct can facilitate expression of the heavy chain polypeptide and/or the light chain polypeptide.

When arrangement 1 as described above is utilized, the first recombinant nucleic acid sequence construct can facilitate the expression of the heavy chain polypeptide and the second recombinant nucleic acid sequence construct can facilitate expression of the light chain polypeptide. When arrangement 2 as described above is utilized, the recombinant nucleic acid sequence construct can facilitate the expression of the heavy chain polypeptide and the light chain polypeptide.

Upon expression, for example, but not limited to, in a cell, organism, or mammal, the heavy chain polypeptide and the light chain polypeptide can assemble into the synthetic antibody. In particular, the heavy chain polypeptide and the light chain polypeptide can interact with one another such that assembly results in the synthetic antibody being capable of binding the antigen. In other embodiments, the heavy chain polypeptide and the light chain polypeptide can interact with one another such that assembly results in the synthetic antibody being more immunogenic as compared to an antibody not assembled as described herein. In still other embodiments, the heavy chain polypeptide and the light chain polypeptide can interact with one another such that assembly results in the synthetic antibody being capable of eliciting or inducing an immune response against the antigen.

Vector

The recombinant nucleic acid sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

The one or more vectors can be a heterologous expression construct, which is generally a plasmid that is used to introduce a specific gene into a target cell. Once the expression vector is inside the cell, the heavy chain polypeptide and/or light chain polypeptide that are encoded by the recombinant nucleic acid sequence construct is produced by the cellular-transcription and translation machinery ribosomal complexes. The one or more vectors can express large amounts of stable messenger RNA, and therefore proteins.

Expression Vector

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleic acid sequence construct may be chimeric, meaning that at least one of its components is heterologous with respect to at least one of its other components.

Plasmid

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, Calif.), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego, Calif), which may be used for protein production in Escherichia coli (E. coli). The plasmid may also be p YES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif.), which may be used for protein production in insect cells. The plasmid may also be pcDNAI or pcDNA3 (Invitrogen, San Diego, Calif.), which may be used for protein production in mammalian cells such as Chinese hamster ovary (CHO) cells.

RNA

In one embodiment, the nucleic acid is an RNA molecule. In one embodiment, the RNA molecule is transcribed from a DNA sequence. Accordingly, in one embodiment, the invention provides an RNA molecule encoding one or more of the MAbs or DMAbs. The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded. A RNA molecule useful with the invention may comprise synthetic RNA. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell.

In one embodiment, the RNA is a nucleoside-modified RNA. Nucleoside-modified RNA have particular advantages over non-modified RNA, including for example, increased stability, low or absent innate immunogenicity, and enhanced translation.

Circular and Linear Vector

The one or more vectors may be circular plasmid, which may transform a target cell by integration into the cellular genome or exist extrachromosomally (e.g., autonomous replicating plasmid with an origin of replication). The vector can be pVAX, pcDNA3.0, or provax, or any other expression vector capable of expressing the heavy chain polypeptide and/or light chain polypeptide encoded by the recombinant nucleic acid sequence construct.

Also provided herein is a linear nucleic acid, or linear expression cassette (“LEC”), that is capable of being efficiently delivered to a subject via electroporation and expressing the heavy chain polypeptide and/or light chain polypeptide encoded by the recombinant nucleic acid sequence construct. The LEC may be any linear DNA devoid of any phosphate backbone. The LEC may not contain any antibiotic resistance genes and/or a phosphate backbone. The LEC may not contain other nucleic acid sequences unrelated to the desired gene expression.

The LEC may be derived from any plasmid capable of being linearized. The plasmid may be capable of expressing the heavy chain polypeptide and/or light chain polypeptide encoded by the recombinant nucleic acid sequence construct. The plasmid can be pNP (Puerto Rico/34) or pM2 (New Caledonia/99). The plasmid may be WLV009, pVAX, pcDNA3.0, or provax, or any other expression vector capable of expressing the heavy chain polypeptide and/or light chain polypeptide encoded by the recombinant nucleic acid sequence construct.

The LEC can be perM2. The LEC can be perNP. perNP and perMR can be derived from pNP (Puerto Rico/34) and pM2 (New Caledonia/99), respectively.

Method of Preparing the Vector

Provided herein is a method for preparing the one or more vectors in which the recombinant nucleic acid sequence construct has been placed. After the final subcloning step, the vector can be used to inoculate a cell culture in a large scale fermentation tank, using known methods in the art.

In other embodiments, after the final subcloning step, the vector can be used with one or more electroporation (EP) devices. The EP devices are described below in more detail.

The one or more vectors can be formulated or manufactured using a combination of known devices and techniques, but preferably they are manufactured using a plasmid manufacturing technique that is described in a licensed, co-pending U.S. provisional application U.S. Ser. No. 60/939,792, which was filed on May 23, 2007. In some examples, the DNA plasmids described herein can be formulated at concentrations greater than or equal to 10 mg/mL. The manufacturing techniques also include or incorporate various devices and protocols that are commonly known to those of ordinary skill in the art, in addition to those described in U.S. Ser. No. 60/939792, including those described in a licensed patent, U.S. Pat. No. 7,238,522, which issued on Jul. 3, 2007. The above-referenced application and patent, US Serial No. 60/939,792 and U.S. Pat. No. 7,238,522, respectively, are hereby incorporated in their entirety.

Antibody

In some embodiments, the invention relates to a recombinant nucleic acid sequence encoding a synthetic antibody, a fragment thereof, a variant thereof, or a combination thereof. The antibody can bind or react with an antigen, which is described in more detail below. In some embodiments, the antibody is a DNA encoded monoclonal antibody (DMAb), a fragment thereof, or a variant thereof. In some embodiments the fragment is an ScFv fragment. In some embodiments, the antibody is a DNA encoded bispecific T cell engagers (BiTE), a fragment thereof, or a variant thereof.

In some embodiments, the antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites. Accordingly, the antibody can be the Fab or F(ab′)₂. The Fab can include the heavy chain polypeptide and the light chain polypeptide. The heavy chain polypeptide of the Fab can include the VH region and the CH1 region. The light chain of the Fab can include the VL region and CL region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The antibody can be a bispecific antibody as described below in more detail. The antibody can be a bifunctional antibody as also described below in more detail.

As described above, the antibody can be generated in the subject upon administration of the composition to the subject. The antibody may have a half-life within the subject. In some embodiments, the antibody may be modified to extend or shorten its half-life within the subject. Such modifications are described below in more detail.

The antibody can be defucosylated as described in more detail below.

ScFv Antibody

In one embodiment, the synthetic antibody of the invention is a ScFv DMAb. In one embodiment, ScFv DMAb relates to a Fab fragment without the of CH1 and CL regions. Thus, in one embodiment, the ScFv DMAb relates to a Fab fragment DMAb comprising the VH and VL. In one embodiment, the ScFv DMAb comprises a linker between VH and VL. In one embodiment, the ScFv DMAb is an ScFv-Fc DMAb. In one embodiment, the ScFv-Fc DMAb comprises the VH, VL and the CH2 and CH3 regions. In one embodiment, the ScFv-Fc DMAb comprises a linker between VH and VL. In one embodiment, the ScFv DMAb of the invention has modified expression, stability, half-life, antigen binding, heavy chain-light chain pairing, tissue penetration or a combination thereof as compared to a parental DMAb.

In one embodiment, the ScFv DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher expression than the parental DMAb.

In one embodiment, the ScFv DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher antigen binding than the parental DMAb.

In one embodiment, the ScFv DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold longer half-life than the parental DMAb.

In one embodiment, the ScFv DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher stability than the parental DMAb.

In one embodiment, the ScFv DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater tissue penetration than the parental DMAb.

In one embodiment, the ScFv DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater heavy chain-light chain pairing than the parental DMAb.

Monoclonal Antibodies

In one embodiment, the synthetic antibody may be an intact monoclonal antibody, an immunologically active fragment (e.g., a Fab or (Fab)₂ fragment), a monoclonal antibody heavy chain, or a monoclonal antibody light chain.

The antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can treat, prevent, and/or protect against disease, such as an infection or cancer, in the subject administered a composition of the invention. The antibody, by binding the antigen, can treat, prevent, and/or protect against disease in the subject administered the composition. The antibody can promote survival of the disease in the subject administered the composition. In one embodiment, the antibody can provide increased survival of the disease in the subject over the expected survival of a subject having the disease who has not been administered the antibody. In various embodiments, the antibody can provide at least about a 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or a 100% increase in survival of the disease in subjects administered the composition over the expected survival in the absence of the composition. In one embodiment, the antibody can provide increased protection against the disease in the subject over the expected protection of a subject who has not been administered the antibody. In various embodiments, the antibody can protect against disease in at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% of subjects administered the composition over the expected protection in the absence of the composition.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites. Accordingly, the antibody can be the Fab or F(ab′)₂. The Fab can include the heavy chain polypeptide and the light chain polypeptide. The heavy chain polypeptide of the Fab can include the VH region and the CH1 region. The light chain of the Fab can include the VL region and CL region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The antibody can be a bispecific antibody as described herein in more detail. The antibody can be a bifunctional antibody as also described herein in more detail.

As described above, the antibody can be generated in the subject upon administration of the composition to the subject. The antibody may have a half-life within the subject. In some embodiments, the antibody may be modified to extend or shorten its half-life within the subject. Such modifications are described herein in more detail.

The antibody can be defucosylated as described in more detail herein.

The antibody may be modified to reduce or prevent antibody-dependent enhancement (ADE) of disease associated with the antigen as described in more detail herein.

Bispecific Antibody

The recombinant nucleic acid sequence can encode a bispecific antibody, a fragment thereof, a variant thereof, or a combination thereof. The bispecific antibody can bind or react with two antigens, for example, two of the antigens described herein in more detail. The bispecific antibody can be comprised of fragments of two of the antibodies described herein, thereby allowing the bispecific antibody to bind or react with two desired target molecules, which may include the antigen, which is described herein in more detail, a ligand, including a ligand for a receptor, a receptor, including a ligand-binding site on the receptor, a ligand-receptor complex, and a marker, including a cancer marker.

Bispecific T cell Engager

As described above, the recombinant nucleic acid sequence can encode a bispecific T cell engager (BiTE), a fragment thereof, a variant thereof, or a combination thereof. The antigen targeting domain of the BiTE can bind or react with the antigen, which is described in more detail below.

The antigen targeting domain of the BiTE may comprise an antibody , a fragment thereof, a variant thereof, or a combination thereof. The antigen targeting domain of the BiTE may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding domain, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites. Accordingly, the antigen targeting domain of the BiTE can be the Fab or F(ab′)₂. The Fab can include the heavy chain polypeptide and the light chain polypeptide. The heavy chain polypeptide of the Fab can include the VH region and the CH1 region. The light chain of the Fab can include the VL region and CL region.

The antigen targeting domain of the BiTE can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antigen targeting domain of the BiTE can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

In one embodiment, at least one of the antigen binding domaing and the immune cell engaging domain of the DBiTE of the invention is a ScFv DNA encoded monoclonal antibody (ScFv DMAb) as described in detail above.

Bifunctional Antibody

The recombinant nucleic acid sequence can encode a bifunctional antibody, a fragment thereof, a variant thereof, or a combination thereof. The bifunctional antibody can bind or react with the antigen described herein. The bifunctional antibody can also be modified to impart an additional functionality to the antibody beyond recognition of and binding to the antigen. Such a modification can include, but is not limited to, coupling to factor H or a fragment thereof. Factor H is a soluble regulator of complement activation and thus, may contribute to an immune response via complement-mediated lysis (CML).

Extension of Antibody Half-Life

As described above, the antibody may be modified to extend or shorten the half-life of the antibody in the subject. The modification may extend or shorten the half-life of the antibody in the serum of the subject.

The modification may be present in a constant region of the antibody. The modification may be one or more amino acid substitutions in a constant region of the antibody that extend the half-life of the antibody as compared to a half-life of an antibody not containing the one or more amino acid substitutions. The modification may be one or more amino acid substitutions in the CH2 domain of the antibody that extend the half-life of the antibody as compared to a half-life of an antibody not containing the one or more amino acid substitutions.

In some embodiments, the one or more amino acid substitutions in the constant region may include replacing a methionine residue in the constant region with a tyrosine residue, a serine residue in the constant region with a threonine residue, a threonine residue in the constant region with a glutamate residue, or any combination thereof, thereby extending the half-life of the antibody.

In other embodiments, the one or more amino acid substitutions in the constant region may include replacing a methionine residue in the CH2 domain with a tyrosine residue, a serine residue in the CH2 domain with a threonine residue, a threonine residue in the CH2 domain with a glutamate residue, or any combination thereof, thereby extending the half-life of the antibody.

Defucosylation

The recombinant nucleic acid sequence can encode an antibody that is not fucosylated (i.e., a defucosylated antibody or a non-fucosylated antibody), a fragment thereof, a variant thereof, or a combination thereof. Fucosylation includes the addition of the sugar fucose to a molecule, for example, the attachment of fucose to N-glycans, O-glycans and glycolipids. Accordingly, in a defucosylated antibody, fucose is not attached to the carbohydrate chains of the constant region. In turn, this lack of fucosylation may improve FcγRIIIa binding and antibody directed cellular cytotoxic (ADCC) activity by the antibody as compared to the fucosylated antibody. Therefore, in some embodiments, the non-fucosylated antibody may exhibit increased ADCC activity as compared to the fucosylated antibody.

The antibody may be modified so as to prevent or inhibit fucosylation of the antibody. In some embodiments, such a modified antibody may exhibit increased ADCC activity as compared to the unmodified antibody. The modification may be in the heavy chain, light chain, or a combination thereof. The modification may be one or more amino acid substitutions in the heavy chain, one or more amino acid substitutions in the light chain, or a combination thereof.

Reduced ADE Response

The antibody may be modified to reduce or prevent antibody-dependent enhancement (ADE) of disease associated with the antigen, but still neutralize the antigen.

In some embodiments, the antibody may be modified to include one or more amino acid substitutions that reduce or prevent binding of the antibody to FcγR1a. The one or more amino acid substitutions may be in the constant region of the antibody. The one or more amino acid substitutions may include replacing a leucine residue with an alanine residue in the constant region of the antibody, i.e., also known herein as LA, LA mutation or LA substitution. The one or more amino acid substitutions may include replacing two leucine residues, each with an alanine residue, in the constant region of the antibody and also known herein as LALA, LALA mutation, or LALA substitution. The presence of the LALA substitutions may prevent or block the antibody from binding to FcγR1a, and thus, the modified antibody does not enhance or cause ADE of disease associated with the antigen, but still neutralizes the antigen.

### 3. METHOD OF GENERATING THE SYNTHETIC ANTIBODY

The present invention also relates a method of generating the synthetic antibody. The method can include administering the composition to the subject in need thereof by using the method of delivery described in more detail herein. Accordingly, the synthetic antibody is generated in the subject or in vivo upon administration of the composition to the subject.

The method can also include introducing the composition into one or more cells, and therefore, the synthetic antibody can be generated or produced in the one or more cells. The method can further include introducing the composition into one or more tissues, for example, but not limited to, skin and muscle, and therefore, the synthetic antibody can be generated or produced in the one or more tissues.

### 4. CHECKPOINT INHIBITORS

In some embodiments, the composition of the invention may comprise a checkpoint inhibitor. The checkpoint inhibitor(s), the inhibitor of B cell maturation and synthetic antibody, or recombinant nucleic acid molecule encoding the same, of the composition can be administered together or separately to the subject in need thereof, in nucleic acid or polypeptide forms. In some instances, the checkpoint inhibitor(s) can be administered separately from the inhibitor of B cell maturation and synthetic antibody, or recombinant nucleic acid molecule encoding the same, of the composition.

Checkpoint inhibitors can be any antagonist to the various immune checkpoints, and may be antibodies that block immune checkpoints. The antibodies can be a protein including a Fab, monoclonal or polyclonal. The antibodies can also be a DNA expression construct that encodes for and can express functional antibodies. The vaccine, in addition to one or more antigens, can further comprise a PD-1 antibody. The antibody can be a synthetic antibody comprised of DNA sequence encoding at least the variable regions of an immunoglobulin. Such antibody can be generated by identifying or screening for the antibody described herein, which is reactive to or binds the antigen described herein. The method of identifying or screening for the antibody can use the antigen in methodologies known to those skilled in art to identify or screen for the antibody. Such methodologies can include, but are not limited to, selection of the antibody from a library (e.g., phage display) and immunization of an animal followed by isolation and/or purification of the antibody. See for example methods available in Raj an, S., and Sidhu, S., Methods in Enzymology, vol 502, Chapter One “Simplified Synthetic Antibody Libraries (2012), which is incorporated herein in its entirety.

Any antibodies of the invention can also be combined with one or more additional checkpoint inhibitor antibodies, including antibodies against one or more of PD-L1, CTLA-4, LAG-3, GITR, CD40, OX40, TIM-3, 4-1BB, and others. The checkpoint inhibitors can be a known product such as, for example, ipilimumab, tremelimumab, pidilizumab, BMS-936559 (See ClinicalTrials.gov Identifier NCT02028403), MPDL3280A (Roche, see ClinicalTrials.gov Identifier NCT02008227), MDX1105-01 (Bristol Myers Squibb, see ClinicalTrials.gov Identifier NCT00729664), MEDI4736 (MedImmune, See ClinicalTrials.gov Identifier NCT01693562), and MK-3475 (Merck, see ClinicalTrials.gov Identifier NCT02129556).

### 5. ANTIGEN

The synthetic antibody or multivalent antibody of the invention is directed to an antigen or fragment or variant thereof. The antigen can be a nucleic acid sequence, an amino acid sequence, a polysaccharide or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof. The polysaccharide can be a nucleic acid encoded polysaccharide.

The antigen can be from a bacterium. The antigen can be associated with bacterial infection. In one embodiment, the antigen can be a bacterial virulence factor. In one embodiment, the antigent can be associated with Pseudomonas aeruginosa infection.

In one embodiment, the antigen can be a lipooligosaccharide.

In one embodiment, a synthetic antibody of the invention targets two or more antigens. In one embodiment, at least one antigen of a bispecific antibody is selected from the antigens described herein. In one embodiment, the two or more antigens are selected from the antigens described herein.

Foreign Antigens

In some embodiments, the antigen is foreign. A foreign antigen is any non-self substance (i.e., originates external to the subject) that, when introduced into the body, is capable of stimulating an immune response.

Bacterial Antigens

The foreign antigen can be a bacterial antigen or fragment or variant thereof. The bacterium can be from any one of the following phyla: Acidobacteria, Actinobacteria, Aquificae, Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, and Verrucomicrobia.

The bacterium can be a gram positive bacterium or a gram negative bacterium. The bacterium can be an aerobic bacterium or an anerobic bacterium. The bacterium can be an autotrophic bacterium or a heterotrophic bacterium. The bacterium can be a mesophile, a neutrophile, an extremophile, an acidophile, an alkaliphile, a thermophile, a psychrophile, an halophile, or an osmophile.

The bacterium can be an anthrax bacterium, an antibiotic resistant bacterium, a disease causing bacterium, a food poisoning bacterium, an infectious bacterium, Salmonella bacterium, Staphylococcus bacterium, Streptococcus bacterium, or tetanus bacterium. The bacterium can be a mycobacteria, Clostridium tetani, Yersinia pestis, Bacillus anthracis, methicillin-resistant Staphylococcus aureus (MRSA), or Clostridium difficile.

Viral Antigens

The foreign antigen can be a viral antigen, or fragment thereof, or variant thereof. The viral antigen can be from a virus from one of the following families: Adenoviridae, Arenaviridae, Bunyaviridae, Caliciviridae, Coronaviridae, Filoviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, or Togaviridae. The viral antigen can be from human immunodeficiency virus (HIV), Chikungunya virus (CHIKV), dengue fever virus, papilloma viruses, for example, human papillomoa virus (HPV), polio virus, hepatitis viruses, for example, hepatitis A virus (HAV), hepatitis B virus (HBV), hepatitis C virus (HCV), hepatitis D virus (HDV), and hepatitis E virus (HEV), smallpox virus (Variola major and minor), vaccinia virus, influenza virus, rhinoviruses, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus, Ebola fever virus, Marburg virus, measles virus, mumps virus, respiratory syncytial virus (RSV), herpes simplex 1 (oral herpes), herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, lassa virus, arenavirus, or cancer causing virus.

Parasitic Antigens

The foreign antigen can be a parasite antigen or fragment or variant thereof. The parasite can be a protozoa, helminth, or ectoparasite. The helminth (i.e., worm) can be a flatworm (e.g., flukes and tapeworms), a thorny-headed worm, or a round worm (e.g., pinworms). The ectoparasite can be lice, fleas, ticks, and mites.

The parasite can be any parasite causing any one of the following diseases: Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Malaria, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, and Trichuriasis.

The parasite can be Acanthamoeba, Anisakis, Ascaris lumbricoides, Botfly, Balantidium coli, Bedbug, Cestoda (tapeworm), Chiggers, Cochliomyia hominivorax, Entamoeba histolytica, Fasciola hepatica, Giardia lamblia, Hookworm, Leishmania, Linguatula serrata, Liver fluke, Loa loa, Paragonimus—lung fluke, Pinworm, Plasmodium falciparum, Schistosoma, Strongyloides stercoralis, Mite, Tapeworm, Toxoplasma gondii, Trypanosoma, Whipworm, or Wuchereria bancrofti.

Fungal Antigens

The foreign antigen can be a fungal antigen or fragment or variant thereof. The fungus can be Aspergillus species, Blastomyces dermatitidis, Candida yeasts (e.g., Candida albicans), Coccidioides, Cryptococcus neoformans, Cryptococcus gattii, dermatophyte, Fusarium species, Histoplasma capsulatum, Mucoromycotina, Pneumocystis jirovecii, Sporothrix schenckii, Exserohilum, or Cladosporium.

Self Antigens

In some embodiments, the antigen is a self antigen. A self antigen may be a constituent of the subject's own body that is capable of stimulating an immune response. In some embodiments, a self antigen does not provoke an immune response unless the subject is in a disease state, e.g., an autoimmune disease.

Self antigens may include, but are not limited to, cytokines, antibodies against viruses such as those listed above including HIV and Dengue, antigens affecting cancer progression or development, and cell surface receptors or transmembrane proteins.

Tumor antigens are proteins that are produced by tumor cells that elicit an immune response, particularly T-cell mediated immune responses. The selection of the antigen binding moiety of the invention will depend on the particular type of cancer to be treated. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

Illustrative examples of a tumor associated surface antigen are CD10, CD19, CD20, CD22, CD33, Fms-like tyrosine kinase 3 (FLT-3, CD135), chondroitin sulfate proteoglycan 4 (CSPG4, melanoma-associated chondroitin sulfate proteoglycan), Epidermal growth factor receptor (EGFR), Her2neu, Her3, IGFR, CD133, IL3R, fibroblast activating protein (FAP), CDCP1, Derlin1, Tenascin, frizzled 1-10, the vascular antigens VEGFR2 (KDR/FLK1), VEGFR3 (FLT4, CD309), PDGFR-α (CD140a), PDGFR-.beta. (CD140b) Endoglin, CLEC14, Tem1-8, and Tie2. Further examples may include A33, CAMPATH-1 (CDw52), Carcinoembryonic antigen (CEA), Carboanhydrase IX (MN/CA IX), CD21, CD25, CD30, CD34, CD37, CD44v6, CD45, CD133, de2-7 EGFR, EGFRvIII, EpCAM, Ep-CAM, Folate-binding protein, G250, Fms-like tyrosine kinase 3 (FLT-3, CD135), c-Kit (CD117), CSF1R (CD115), HLA-DR, IGFR, IL-2 receptor, IL3R, MCSP (Melanoma-associated cell surface chondroitin sulphate proteoglycane), Muc-1, Prostate-specific membrane antigen (PSMA), Prostate stem cell antigen (PSCA), Prostate specific antigen (PSA), and TAG-72. Examples of antigens expressed on the extracellular matrix of tumors are tenascin and the fibroblast activating protein (FAP).

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

Aspects of the present invention include compositions for enhancing an immune response against an antigen in a subject in need thereof, comprising a synthetic multivalent antibody capable of generating an immune response in the subject, or a biologically functional fragment or variant thereof.

### 6. EXCIPIENTS AND OTHER COMPONENTS OF THE COMPOSITION

The composition may further comprise a pharmaceutically acceptable excipient. The pharmaceutically acceptable excipient can be functional molecules such as vehicles, adjuvants, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example W09324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the composition is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant in addition to the checkpoint inhibitor antibodies of the invention. The additional adjuvant can be other genes that are expressed in an alternative plasmid or are delivered as proteins in combination with the plasmid above in the composition. The adjuvant may be selected from the group consisting of: α-interferon(IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, PD-1, IL-10, IL-12, IL-18, or a combination thereof.

Other genes that can be useful as adjuvants in addition to the antibodies of the invention include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The composition may further comprise a genetic facilitator agent as described in U.S. Ser. No. 021,579 filed Apr. 1, 1994, which is fully incorporated by reference.

The composition may comprise DNA at quantities of from about 1 nanogram to 100 milligrams; about 1 microgram to about 10 milligrams; or preferably about 0.1 microgram to about 10 milligrams; or more preferably about 1 milligram to about 2 milligram. In some preferred embodiments, composition according to the present invention comprises about 5 nanogram to about 1000 micrograms of DNA. In some preferred embodiments, composition can contain about 10 nanograms to about 800 micrograms of DNA. In some preferred embodiments, the composition can contain about 0.1 to about 500 micrograms of DNA. In some preferred embodiments, the composition can contain about 1 to about 350 micrograms of DNA. In some preferred embodiments, the composition can contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligram, from about 5 nanogram to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of DNA.

The composition can be formulated according to the mode of administration to be used. An injectable pharmaceutical composition can be sterile, pyrogen free and particulate free. An isotonic formulation or solution can be used.

Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

### 7. METHOD OF VACCINATION

The present invention is also directed to a method of increasing an immune response in a subject. Increasing the immune response can be used to treat and/or prevent disease in the subject. The method can include administering the herein disclosed vaccine to the subject. The subject administered the vaccine can have an increased or boosted immune response as compared to a subject administered the antigen alone. In some embodiments, the immune response can be increased by about 0.5-fold to about 15-fold, about 0.5-fold to about 10-fold, or about 0.5-fold to about 8-fold. Alternatively, the immune response in the subject administered the vaccine can be increased by at least about 0.5-fold, at least about 1.0-fold, at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, or at least about 15.0-fold.

In still other alternative embodiments, the immune response in the subject administered the vaccine can be increased about 50% to about 1500%, about 50% to about 1000%, or about 50% to about 800%. In other embodiments, the immune response in the subject administered the vaccine can be increased by at least about 50%, at least about 100%, at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, at least about 1050%, at least about 1100%, at least about 1150%, at least about 1200%, at least about 1250%, at least about 1300%, at least about 1350%, at least about 1450%, or at least about 1500%.

The vaccine dose can be between 1 μg to 10 mg active component/kg body weight/time, and can be 20 μg to 10 mg component/kg body weight/time. The vaccine can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of vaccine doses for effective treatment can be 1, 2, 3, 4, 5, 6, 7, 8, 9, or 10.

### 8. METHOD OF DELIVERY OF THE COMPOSITION

The present invention also relates to a method of delivering the composition to the subject in need thereof. The method of delivery can include, administering the composition to the subject. Administration can include, but is not limited to, DNA injection with and without in vivo electroporation, liposome mediated delivery, and nanoparticle facilitated delivery.

The mammal receiving delivery of the composition may be human, primate, non-human primate, cow, cattle, sheep, goat, antelope, bison, water buffalo, bison, bovids, deer, hedgehogs, elephants, llama, alpaca, mice, rats, and chicken.

The composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal, intranasal, intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

a. Electroporation

Administration of the composition via electroporation may be accomplished using electroporation devices that can be configured to deliver to a desired tissue of a mammal, a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation may be accomplished using an in vivo electroporation device, for example CELLECTRA EP system (Inovio Pharmaceuticals, Plymouth Meeting, Pa.) or Elgen electroporator (Inovio Pharmaceuticals, Plymouth Meeting, Pa.) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μs, 20 μs, 10 μs or 1 μs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the composition of the present invention, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the composition include those provided in co-pending and co-owned U.S. patent application, Ser. No. 11/874072, filed Oct. 17, 2007, which claims the benefit under 35 USC 119(e) to U.S. Provisional Applications Ser. Nos. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference.

The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes The electrodes described in U.S. Pat. No. 7,245,963 and U.S. Patent Pub. 2005/005263 are preferably 20 mm long and 21 gauge.

Additionally, contemplated in some embodiments, that incorporate electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to method of injecting DNA are contemplated herein. The above-patents are incorporated by reference in their entirety.

### 9. METHOD OF TREATMENT

Also provided herein is a method of treating, protecting against, and/or preventing disease in a subject in need thereof by administering a combination of an inhibitor of B cell maturation or function and a composition for generating a synthetic antibody in the subject. In some embodiments, the method can include administering a single composition comprising an inhibitor of B cell maturation or function and a synthetic antibody, or recombinant nucleic acid molecule encoding the same, to the subject. In some embodiments, the method can include administering a combination of a first composition comprising an inhibitor of B cell maturation or function and second composition comprising a synthetic antibody, or recombinant nucleic acid molecule encoding the same, to the subject. Administration of the composition to the subject can be done using the method of delivery described above.

In certain embodiments, the invention provides a method of treating protecting against, and/or preventing a disease or disorder associated with a target of the synthetic antibody or synthetic multivalent antibody. In various embodiments, the disease or disorder is a bacterial infection, a viral infection, a fungal infection, a disease or disorder associated with a parasite, or a disease or disorder associated with a self antigen, including, but not limited to, cancer.

Upon generation of the synthetic antibody in the subject, the synthetic antibody can bind to or react with the antigen. Such binding can neutralize the antigen, block recognition of the antigen by another molecule, for example, a protein or nucleic acid, and elicit or induce an immune response to the antigen, thereby treating, protecting against, and/or preventing the disease associated with the antigen in the subject.

The synthetic antibody can treat, prevent, and/or protect against disease in the subject administered the composition. The synthetic antibody by binding the antigen can treat, prevent, and/or protect against disease in the subject administered the composition. The synthetic antibody can promote survival of the disease in the subject administered the composition. In one embodiment, the synthetic antibody can provide increased survival of the disease in the subject over the expected survival of a subject having the disease who has not been administered the synthetic antibody. In various embodiments, the synthetic antibody can provide at least about a 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or a 100% increase in survival of the disease in subjects administered the composition over the expected survival in the absence of the composition. In one embodiment, the synthetic antibody can provide increased protection against the disease in the subject over the expected protection of a subject who has not been administered the synthetic antibody. In various embodiments, the synthetic antibody can protect against disease in at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% of subjects administered the composition over the expected protection in the absence of the composition.

The composition dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

### 10. USE IN COMBINATION WITH ANTIBIOTICS

The present invention also provides a method of treating, protecting against, and/or preventing disease in a subject in need thereof by administering a combination of the synthetic antibody and a therapeutic antibiotic agent.

The synthetic antibody and an antibiotic agent may be administered using any suitable method such that a combination of the synthetic antibody and antibiotic agent are both present in the subject. In one embodiment, the method may comprise administration of a first composition comprising a synthetic antibody of the invention by any of the methods described in detail above and administration of a second composition comprising an antibiotic agent less than 1, less than 2, less than 3, less than 4, less than 5, less than 6, less than 7, less than 8, less than 9 or less than 10 days following administration of the synthetic antibody. In one embodiment, the method may comprise administration of a first composition comprising a synthetic antibody of the invention by any of the methods described in detail above and administration of a second composition comprising an antibiotic agent more than 1, more than 2, more than 3, more than 4, more than 5, more than 6, more than 7, more than 8, more than 9 or more than 10 days following administration of the synthetic antibody. In one embodiment, the method may comprise administration of a first composition comprising an antibiotic agent and administration of a second composition comprising a synthetic antibody of the invention by any of the methods described in detail above less than 1, less than 2, less than 3, less than 4, less than 5, less than 6, less than 7, less than 8, less than 9 or less than 10 days following administration of the antibiotic agent. In one embodiment, the method may comprise administration of a first composition comprising an antibiotic agent and administration of a second composition comprising a synthetic antibody of the invention by any of the methods described in detail above more than 1, more than 2, more than 3, more than 4, more than 5, more than 6, more than 7, more than 8, more than 9 or more than 10 days following administration of the antibiotic agent. In one embodiment, the method may comprise administration of a first composition comprising a synthetic antibody of the invention by any of the methods described in detail above and a second composition comprising an antibiotic agent concurrently. In one embodiment, the method may comprise administration of a first composition comprising a synthetic antibody of the invention by any of the methods described in detail above and a second composition comprising an antibiotic agent concurrently. In one embodiment, the method may comprise administration of a single composition comprising a synthetic antibody of the invention and an antibiotic agent.

Non-limiting examples of antibiotics that can be used in combination with the synthetic antibody of the invention include aminoglycosides (e.g., gentamicin, amikacin, tobramycin), quinolones (e.g., ciprofloxacin, levofloxacin), cephalosporins (e.g., ceftazidime, cefepime, cefoperazone, cefpirome, ceftobiprole), antipseudomonal penicillins: carboxypenicillins (e.g., carbenicillin and ticarcillin) and ureidopenicillins (e.g., mezlocillin, azlocillin, and piperacillin), carbapenems (e.g., meropenem, imipenem, doripenem), polymyxins (e.g., polymyxin B and colistin) and monobactams (e.g., aztreonam).

### 11. CANCER THERAY

In one embodiment, the invention has multiple provides methods of treating or preventing cancer, or of treating and preventing growth or metastasis of tumors. Related aspects, illustrated of the invention provide methods of preventing, aiding in the prevention, and/or reducing metastasis of hyperplastic or tumor cells in an individual.

One aspect of the invention provides a method of inhibiting metastasis in an individual in need thereof, the method comprising administering to the individual an effective amount of a nucleic acid molecule encoding a multivalent antibody of the invention, wherein the multivalent antibody is specific for the cancer to be treated. The invention further provides a method of inhibiting metastasis in an individual in need thereof, the method comprising administering to the individual an effective metastasis-inhibiting amount of a nucleic acid molecule encoding a multivalent antibody of the invention, wherein the multivalent antibody is specific for the cancer to be treated.

In some embodiments of treating or preventing cancer, or of treating and preventing metastasis of tumors in an individual in need thereof, a second agent is administered to the individual, such as an antineoplastic agent. In some embodiments, the second agent comprises a second metastasis-inhibiting agent, such as a plasminogen antagonist, or an adenosine deaminase antagonist. In other embodiments, the second agent is an angiogenesis inhibiting agent.

The compositions of the invention can be used to prevent, abate, minimize, control, and/or lessen cancer in humans and animals. The compositions of the invention can also be used to slow the rate of primary tumor growth. The compositions of the invention when administered to a subject in need of treatment can be used to stop the spread of cancer cells. As such, an effective amount of a nucleic acid molecule encoding a multivalent antibody of the invention, wherein the multivalent antibody is specific for the cancer to be treated can be administered as part of a combination therapy with one or more drugs or other pharmaceutical agents. When used as part of the combination therapy, the decrease in metastasis and reduction in primary tumor growth afforded by the compositions of the invention allows for a more effective and efficient use of any pharmaceutical or drug therapy being used to treat the patient. In addition, control of metastasis by the compositions of the invention affords the subject a greater ability to concentrate the disease in one location.

In one embodiment, the invention provides methods for preventing metastasis of malignant tumors or other cancerous cells as well as to reduce the rate of tumor growth. The methods comprise administering an effective amount of a nucleic acid molecule encoding a multivalent antibody of the invention, wherein the multivalent antibody is specific for the cancer to be treated, to a subject diagnosed with a malignant tumor or cancerous cells or to a subject having a tumor or cancerous cells.

The following are non-limiting examples of cancers that can be treated by the methods and compositions of the invention: Acute Lymphoblastic; Acute Myeloid Leukemia; Adrenocortical Carcinoma; Adrenocortical Carcinoma, Childhood; Appendix Cancer; Basal Cell Carcinoma; Bile Duct Cancer, Extrahepatic; Bladder Cancer; Bone Cancer; Osteosarcoma and Malignant Fibrous Histiocytoma; Brain Stem Glioma, Childhood; Brain Tumor, Adult; Brain Tumor, Brain Stem Glioma, Childhood; Brain Tumor, Central Nervous System Atypical Teratoid/Rhabdoid Tumor, Childhood; Central Nervous System Embryonal Tumors; Cerebellar Astrocytoma; Cerebral Astrocytotna/Malignant Glioma; Craniopharyngioma; Ependymoblastoma; Ependymoma; Medulloblastoma; Medulloepithelioma; Pineal Parenchymal Tumors of intermediate Differentiation; Supratentorial Primitive Neuroectodermal Tumors and Pineoblastoma; Visual Pathway and Hypothalamic Glioma; Brain and Spinal Cord Tumors; Breast Cancer; Bronchial Tumors; Burkitt Lymphoma; Carcinoid Tumor; Carcinoid Tumor, Gastrointestinal; Central Nervous System Atypical Teratoid/Rhabdoid Tumor; Central Nervous System Embryonal Tumors; Central Nervous System Lymphoma; Cerebellar Astrocytoma Cerebral Astrocytoma/Malignant Glioma, Childhood; Cervical Cancer; Chordoma, Childhood; Chronic Lymphocytic Leukemia; Chronic Myelogenous Leukemia; Chronic Myeloproliferative Disorders; Colon Cancer; Colorectal Cancer; Craniopharyngioma; Cutaneous T-Cell Lymphoma; Esophageal Cancer; Ewing Family of Tumors; Extragonadal Germ Cell Tumor; Extrahepatic Bile Duct Cancer; Eye Cancer, intraocular Melanoma; Eye Cancer, Retinoblastoma; Gallbladder Cancer; Gastric (Stomach) Cancer; Gastrointestinal Carcinoid Tumor; Gastrointestinal Stromal Tumor (GIST); Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer; Histiocytosis, Langerhans Cell; Hodgkin Lymphoma; Hypopharyngeal Cancer; Hypothalamic and Visual Pathway Glioma; intraocular Melanoma; Islet Cell Tumors; Kidney (Renal Cell) Cancer; Langerhans Cell Histiocytosis; Laryngeal Cancer; Leukemia, Acute Lymphoblastic; Leukemia, Acute Myeloid; Leukemia, Chronic Lymphocytic; Leukemia, Chronic Myelogenous; Leukemia, Hairy Cell; Lip and Oral Cavity Cancer; Liver Cancer; Lung Cancer, Non-Small Cell; Lung Cancer, Small Cell; Lymphoma, AIDS-Related; Lymphoma, Burkitt; Lymphoma, Cutaneous T-Cell; Lymphoma, Hodgkin; Lymphoma, Non-Hodgkin; Lymphoma, Primary Central Nervous System; Macroglobulinemia, Waldenstrom; Malignant Fibrous Histiocvtoma of Bone and Osteosarcoma; Medulloblastoma; Melanoma; Melanoma, intraocular (Eye); Merkel Cell Carcinoma; Mesothelioma; Metastatic Squamous Neck Cancer with Occult Primary; Mouth Cancer; Multiple Endocrine Neoplasia Syndrome, (Childhood); Multiple Myeloma/Plasma Cell Neoplasm; Mycosis; Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Neuroblastoma; Non-Small Cell Lung Cancer; Oral Cancer; Oral Cavity Cancer; Oropharyngeal Cancer; Osteosarcoma and Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer, Islet Cell Tumors; Papillomatosis; Parathyroid Cancer; Penile Cancer; Pharyngeal Cancer; Pheochromocytoma; Pineal Parenchymal Tumors of Intermediate Differentiation; Pineoblastoma and Supratentorial Primitive Neuroectodermal Tumors; Pituitary Tumor; Plasma Celt Neoplasm/Multiple Myeloma; Pleuropulmonary Blastoma; Primary Central Nervous System Lymphoma; Prostate Cancer; Rectal Cancer; Renal Cell (Kidney) Cancer; Renal Pelvis and Ureter, Transitional Cell Cancer; Respiratory Tract Carcinoma Involving the NUT Gene on Chromosome 15; Retinoblastoma; Rhabdomyosarcoma; Salivary Gland Cancer; Sarcoma, Ewing Family of Tumors; Sarcoma, Kaposi; Sarcoma, Soft Tissue; Sarcoma, Uterine; Sezary Syndrome; Skin Cancer (Nonmelanoma); Skin Cancer (Melanoma); Skin Carcinoma, Merkel Cell; Small Cell Lung Cancer; Small Intestine Cancer; Soft Tissue Sarcoma; Squamous Cell Carcinoma, Squamous Neck Cancer with Occult Primary, Metastatic; Stomach (Gastric) Cancer; Supratentorial Primitive Neuroectodermal Tumors; T-Cell Lymphoma, Cutaneous; Testicular Cancer; Throat Cancer; Thymoma and Thymic Carcinoma; Thyroid Cancer; Transitional Cell Cancer of the Renal Pelvis and Ureter; Trophoblastic Tumor, Gestational; Urethral Cancer; Uterine Cancer, Endometrial; Uterine Sarcoma; Vaginal Cancer; Vulvar Cancer; Waldenstrom Macroglobulinemia; and Wilms Tumor.

In one embodiment, the invention provides a method to treat cancer metastasis comprising treating the subject prior to, concurrently with, or subsequently to the treatment with a composition of the invention, with a complementary therapy for the cancer, such as surgery, chemotherapy, chemotherapeutic agent, radiation therapy, or hormonal therapy or a combination thereof.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The compounds of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the invention include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the compositions of the invention include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

### 12. GENERATION OF SYNTHETIC ANTIBODIES IN VITRO AND EX VIVO

In one embodiment, the synthetic antibody or synthetic multivalent antibody is generated in vitro or ex vivo. For example, in one embodiment, a nucleic acid encoding a synthetic antibody can be introduced and expressed in an in vitro or ex vivo cell. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, N.Y.). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

The present invention is further illustrated in the following Examples. It should be understood that these Examples, while indicating exemplary embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### 13. EXAMPLES

Example 1

Example 1: Transient CD40L Immune Blockade Prevents Development of Anti-Drug Antibodies following In Vivo Delivery of Xenogenic Human IgG Plasmid DNA-Encoded Antibodies (DMAbs) in Mice

Synthetic non-viral nucleic acids (plasmid DNA and mRNA) and viral adeno-associated virus vectors (AAV) are rapidly advancing platforms for gene-encoded in vivo delivery of monoclonal antibody (mAb) biologics. Like recombinant biologics, the development of anti-drug antibodies (ADA) against mAbs can dramatically impact biologic efficacy, resulting in lower in vivo potency and duration, as well as impaired re-administration. Utilizing a synthetic DNA-encoded mAb (DMAb) platform it was shown that CD4+ and CD8+ T cell depletion prevents the development of ADA. T cell depletion positively enhances long-term expression of xenogenic human IgG DMAbs in a mouse host, resulting in expression lasting >365 days. However, T cell depletion is not ideal for clinical treatment regimens and alternative solutions to overcome ADA are important for the translation of gene-encoded mAb platforms. To address this, transient blockade of early innate immune signals in parallel with DMAb delivery including strategies that target early co-stimulation and downstream intracellular signaling pathways was investigated. Mice (n=5 mice/group) were administered DMAb in combination with rapamycin, CTLA4-Ig, or anti-CD40L (anti-CD154). Daily administration of rapamycin and CTLA4-Ig both delayed development of ADA, however mouse anti-human DMAb antibodies developed when treatment was stopped. Interestingly, transient blockade mediated by anti-CD40L resulted in suppression of ADA. A single administration provided enhanced human IgG1 DMAb expression with extended duration in circulation of >365 days, similar to T cell depletion. These results were consistent for DMAbs targeting Pseudomonas aeruginosa, influenza virus, and HIV. Taken together, these results demonstrate that CD40L blockade is a simple approach with potential applications for clinical translation. This is an important step to mask gene-encoded antibodies from immune surveillance, with potential application for other gene therapies.

### Example 2: Potent In Vivo Enhancement and Extension of Circulating DMAbs in Serum by Transient Blockade of CD40L

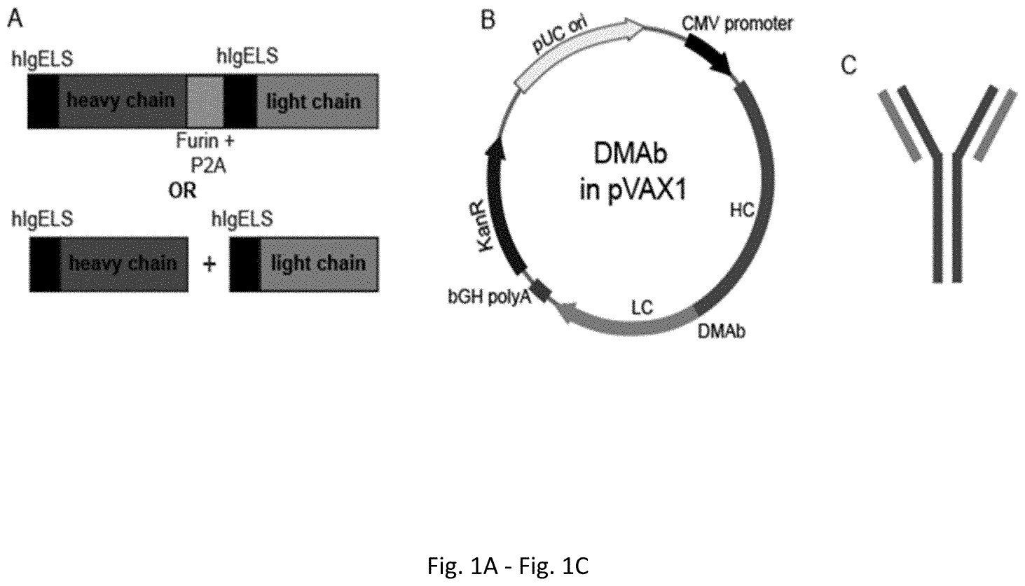

Here, it is demonstrated that modulation of early immune signaling can mask ADA against xenogeneic DMAbs, prolonging expression in vivo. The three-signal model for immune activation (FIG. 1A-FIG. 1C) initiates with antigen presentation by dendritic cells on MHC-peptide complexes (Signal 1), followed by a second signal provided by co-stimulatory molecules to activate T cells (Signal 2). Lastly, signaling events including cytokine exposure and mTOR pathway activation contribute to T cell polarization (Signal 3). Understanding of these pathways and modulation of both Signal 2 and Signal 3 pathways have led to significant therapeutic advances in immunotherapies for autoimmunity and transplantation. For instance, the use of inhibitory CTLA4-Ig binds with higher affinity to CD80 (B7.1) and CD86 (B7.2) on antigen presenting cells (APCs) than co-stimulatory molecule CD28, providing remarkably effective second signal blocking. Alternatively, second signal modulation provided by CD28, 4-1BB or ICOS can enhance CART cell in vivo function (Guedan, S. et al., 2018, JCI Insight, 3(1)). The blockade of CTLA4 or PD-1/PD-L1 regulatory checkpoint molecules can broadly stimulate anti-tumor immunity (Mahoney, K. M. et al., 2015, Nat. Rev. Drug Discov., 14(8):561-584). As well, the blockade of the inflammatory cytokine signaling (Signal 3) as TNFα or IL-1R for the treatment of autoimmune diseases have changed the standard of care of multiple diseases (Singh, J. A. et al., 2009, Cochrane Database Syst. Rev., 4: CD007848). Parallel interactions between CD40 on CD4+ T cells and its ligand on APCs, CD40L(CD154), play important roles in the immune activation pathway (Elgueta, R. et al., 2009, Immunol. Rev., 229(1):152-172). Antibody-mediated blockade of this pathway has been studied in preclinical models and clinical trials for the treatment of transplantation (Kim, S. C. et al., 2017, Am. J. Transplant, 17(5):1182-1192; Cordoba, F. et al., Am. J. Transplant, 15(11):2825-2836) and autoimmune diseases (Boumpas, D. T. et al., 2003,Arthritis Rheum., 48(3):719-727; Watanabe, M. et al., 2013, Am. J. Transplant, 13(8):1976-1988) (FIG. 1A-FIG. 1C). Glucocorticoids and mTOR pathway inhibitors act more downstream to suppress cytokine signaling and polarization of the immune response. These have proved successful in both autoimmunity and transplantation.

Here, the impact of gene-encoded, synthetic DMAb delivery in combination with immune regulation using clinically translatable antibody biologics and synthetic drugs to prevent development of ADA and prolong in vivo expression was studied. DMAb co-delivery was systematically evaluated with glucocorticosteroid (methylprednisolone), an mTOR pathway inhibitor (rapamycin), and biologics CTLA4-Ig and anti-CD40L (anti-CD154). The data demonstrate that anti-CD40L at the time of delivery prevents the formation of anti-DMAb antibodies. DMAb just entering first-in-human clinical trials (NCT03831503). The data support further study as a potentially translatable approach for humans for DMAb delivery and other gene-encoded antibody platforms.

The materials and methods employed in these experiments are now described.

### Animals and Cell Lines

C57BL/6, BALB/c and B6.129S2-Ighmtm1Cgn/J (muMt−) mice were purchased from Jackson or Charles River Laboratories. Animal experiments were approved by the Institutional Animal Care and Use Committee at The Wistar Institute.

### Immune Suppressants

Rapamycin: Each mouse received a 0.5 mg/kg dose of rapamycin daily by oral gavage. Methylprednisolone: Each mouse received a 10 mg/kg dose of Depomedrol by intramuscular injection.

### Depletion Antibodies

CD40L blockade was performed with anti-CD40L antibody MR1 (BioXCell). Mice were injected with 500 ug of purified antibody intraperitoneally on days −2, 0 and +7 or on the same day of DMAb administration. T-cells were depleted using anti-CD4 clone GK1.5 (200 ug at days 0; BioXCell) and anti-CD8 clone YTS 169.4 (200 ug at day 0; BioXCell).

### Design of DMAbs

### Plasmid DNA Constructs for DMAb and DNA Vaccines

Construction of DNA plasmids encoding anti-Pseudomonas aeruginosa DMAb-V2L2, anti-Ebolavirus DMAb-11, anti-influenza FluA DMAb, and anti-HIV DMAb-PGT128 were previous described (Elliott, S. T. C. et al., 2017, NPJ Vaccines, 2(1): 1-9; Patel A. et al., 2017, Nat. Commun., 8(1):1-11; Patel, A. et al., 2018, Cell Rep., 25(7):1982-1993; Wise, M. C. et al., 2019, J. Clin. Invest., 130(2)). Mice were administered DMAbs by injection of 100 ug-200 ug of DNA in the tibialis anterior (TA) and quadriceps muscles followed by electroporation using the CELLECTRA 3P (Inovio). For vaccination, mice were immunized with bug influenza H3 HA DNA vaccine (ConH3HA-1, ConH3HA-2, ConH3HA-3, and ConH3HA-4 (3)) in the TA muscle followed by electroporation, as previously described (Elliott, S. et al., 2018, Hum. Gene. Ther., 29(9):1044-1055).

Briefly, antibody genes were codon-optimized for mammalian expression in mouse/human and synthesized both heavy and light chain antibody DNA sequences and sub-cloned these into either a single mammalian expression plasmid (PGX0001, a modified pVax plasmid) construct separated by a furin and porcine teschovirus-1 2A (P2A) cleavage site, or into separate modified pVaxl plasmids which were co-mixed prior to injection. A human IgG leader sequence was added to both heavy- and light-chain transgenes. A human IgE leader sequence was added for the influenza A H3 DNA vaccine consensus sequences.

### DMAb Quantification ELISA

ELISA plates were coated with lug/ml of goat anti-human IgG-Fc fragment antibody (Bethyl) overnight at 4° C. The following day, ELISA plates were blocked with PBST-10% FBS for 1 hour at room temperature, washed, incubated for 1 hour at room temperature with the samples diluted in PBST-1%FBS, washed, and incubated at room temperature with HRP conjugated goat anti-human kappa light chain antibody (Bethyl). After 1 h incubation, plates were developed with SIGMAFAST OPD (Sigma Aldrich) and read at 450 nm. A standard curve was generated using purified human IgG/Kappa (Bethyl).

Mouse Anti-DMAb IgG quantification ELISA

ELISA plates were coated with lug/ml of V2L2 antibody overnight at 4° C. The following day, plates were blocked with PBST-10%FBS for 1 hour at room temperature, washed, incubated for 1 hour at room temperature with the samples diluted in PBST −1% FBS, washed, and incubated at room temperature with HRP conjugated goat anti-mouse IgG antibody (Abcam). After 1 h incubation, plates were developed with SIGMAFAST OPD (Sigma Aldrich).

### Influenza A H3 Binding ELISA

Ninety-six well ELISA plates (Nunc MaxiSorp, ThermoFisher) were coated with 2 μg/mL of recombinant antigen HA1 from A/Hong Kong/1/1968 (Immune-Tech) overnight at 4° C., and blocked with 0.5% bovine serum albumin (BSA, MilliporeSigma) in phosphate buffered saline (PBS) for two hours at 25° C. Sera from individual mice were added at a 1:50 starting dilution, with four-fold serial dilutions in 0.5% BSA-T solution for one hour at 25° C. Secondary antibody goat anti-human IgG-heavy-and-light chain (DMAb) or goat anti-mouse IgG-heavy-and-light-chain (DNA vaccine) conjugated to horseradish peroxidase (Millipore Sigma) were added at 1:5,000 in 0.5% BSA-T for one hour, plates were developed 20 minutes with SigmaFast OPD substrate (Millipore Sigma) and stopped with 2 M sulfuric acid. Absorbance was read at a wavelength of 492 nm (Synergy 2, BioTek, Winooski, Vt., USA). Reciprocal endpoint binding titers were calculated according to the method described in Frey, et. al (J Immunol Methods, 1998: 221, 35-41) at a 99.0% confidence level.

### ELISPOT

Splenocytes were harvested and co-incubated with H3 peptide pools (15-mers overlapping by 9 amino acids) for 24 hours. The mouse interferon-y ELISPOT was performed according to the manufacturer's instructions (Mabtech).

### Statistical Analysis

Differences between the means of experimental groups were calculated using a two-tailed unpaired Student's t test or one-way ANOVA where two categorical variables were measured. Repeated measures were analyzed using 2-way ANOVA. Correlation was done with Pearson's test. Error bars represent standard error of the mean. All statistical analyses were done using Graph Pad Prism 7.0. p<0.05 was considered statistically significant.

The results of the experiments are now described.

### DNA-Encoded Monoclonal Antibody (DMAb) Design

DMAbs targeting Pseudomonas aeruginosa, Ebolavirus, influenza virus, and HIV were constructed as previously described (Elliott, S. T. C. et al., 2017, NPJ Vaccines, 2(1): 1-9; Patel A. et al., 2017, Nat. Commun., 8(1):1-11; Patel, A. et al., 2018, Cell Rep., 25(7):1982-1993; Wise, M. C. et al., 2019, J. Clin. Invest., 130(2)). Briefly, monoclonal antibody heavy chains (HC) and light chains (LC) were nucleotide and amino acid optimized and then cloned into an optimized pVaxl expression plasmid, under the control of a human cytomegalovirus (hCMV) promoter and bovine growth hormone (BGH) polyadenylation signal (polyA) (FIG. 1). DMAbs were expressed as a single plasmid, containing furin and P2A cleavage sites, or as two separate plasmids encoding the HC and LC. It was previously shown in mice and non-human primates that DMAb DNA injected intramuscularly followed by adaptive CELLECTRA electroporation, results in in vivo antibody expression at microgram levels in both animal models (Elliott, S. T. C. et al., 2017, NPJ Vaccines, 2(1): 1-9; Patel A. et al., 2017, Nat. Commun., 8(1):1-11; Muthumani, K. et al., 2016, J. Infect. Dis., 214(3):369-378; Wise, M. C. et al., 2019, J. Clin. Invest., 130(2); Muthumani, K. et al., 2017, Cancer Immunol. Immunother., 66(12):1577-1588; Esquivel R. N. et al., 2019, Mol. Ther., 27(5):974-985). However, the development of ADA can impair long-term expression. Although effective, strategies such as CD4+ and CD8+ T cell depletion in mice are unrealistic in the clinic and other solutions to reduce potential anti-DMAb antibodies using translatable drug regimens would be highly useful.

Clearance of Human DMAb from Sera by Anti-DMAb Antibodies

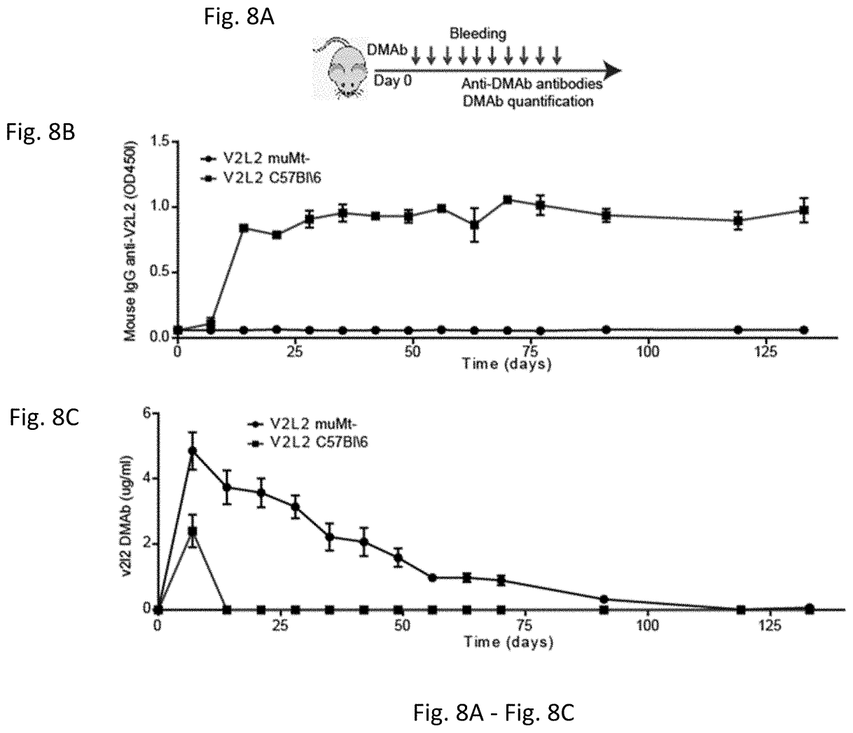

ADA against conventional protein monoclonal antibody therapeutics have been frequently observed and can alter pharmacodynamics and pharmacokinetics of antibodies in sera after administration of mAbs (Gomez-Mantilla, J. D. et al., 2014, J Pharmacokinet. Pharmacodyn., 41(5):523-536). It was previously shown that the development of anti-DMAb ADA is primarily CD4+T cell mediated and MHC Class II dependent (Patel, A. et al., 2018, Cell Rep., 25(7):1982-1993). To further support this data, the expression of a human-IgG1 DMAb-V2L2 in fully immunocompetent versus the MuMt-B cell deficient mouse model was compared (FIG. 8A). As expected, immunocompetent C57Bl/6 mice had increasing levels of ADA starting one week after DMAb administration. However, anti-DMAb antibodies did not develop in the in B-cell deficient C57Bl/6 background (Mu−/−) mice (FIG. 8B), extending the duration of circulating DMAb in serum and underscoring the importance of host anti-DMAb antibodies in the disappearance of heterologous IgG from sera (FIG. 8C).

Development of T Cell Responses following Delivery of Human DMAbs in Mice

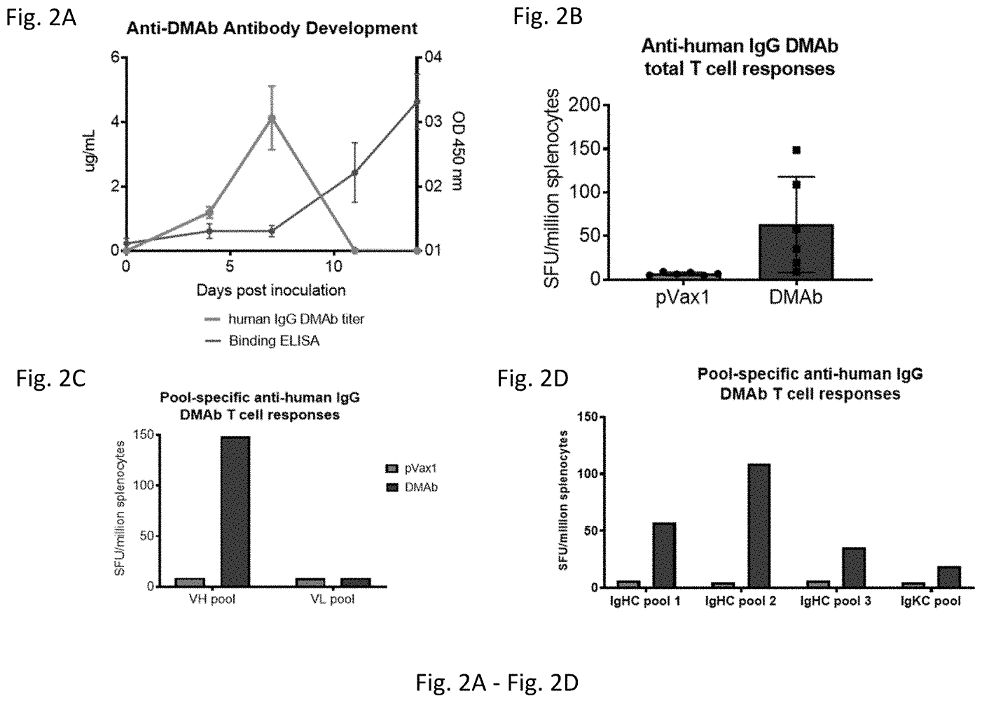

In addition to the development of antibody responses, next the development of T cell responses against human IgG in mice following DMAb administration was evaluated. The clearance of anti-EbolaGP DMAb-11 from mouse sera correlates with an increase in anti-DMAb antibodies, as detected by ELISA (FIG. 2A). T cell responses were assayed by IFNg ELISpot assay from spleens harvest 14 days post-DMAb administration. Splenocytes were re-stimulated with overlapping linear peptide pools corresponding to the entire human IgG Fc and DMAb-specific Fab region. T cell responses were observed in the DMAb administered group (FIG. 2B) and against both the variable VH and VL regions (FIG. 2C) and HC region (FIG. 2D).

### Reducing Injection Site Inflammation and Inhibiting Intracellular Signaling Pathways

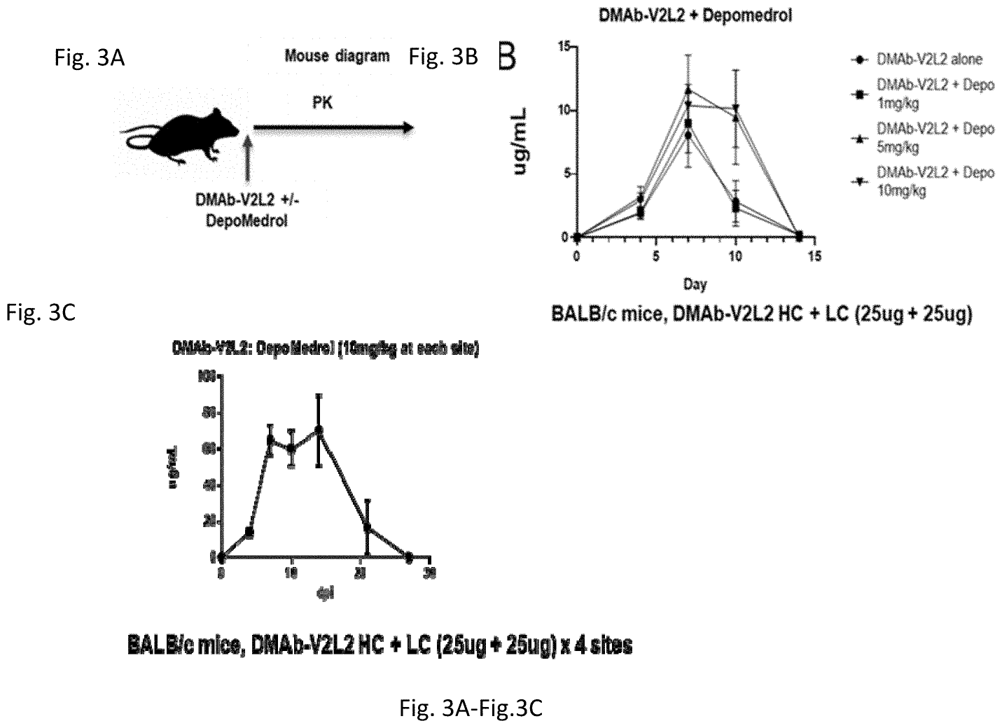

Injection site inflammation can contribute to increased innate immune activation; therefore, the impact of local steroid delivery on the development of anti-DMAb immune responses was evaluated first. Glucocortoids bind to the intracellular glucocorticoid receptor, leading to chaperone binding and translocation into the nucleus. This results in inhibition of pathways such as COX-2 and down-regulation of inflammatory cytokines that may recruit immune cells. First, if a locally delivered, glucorticoid could prevent the development of anti-DMAb immune responses was addressed. Anti-human IgG1 DMAb-V2L2 (two plasmids, 50 ug total DNA) was administered to BALB/c mice alone or in co-formulation with increasing doses of DepoMedrol (methylprednisolone acetate, Zoetis, 1 mg/kg, 5 mg/kg, and 10 mg/kg). Using pharmacokinetic (PK) expression as a benchmark, a delay in the development of anti-DMAb immune responses was observed at the highest 10 mg/kg dose (FIG. 3A, FIG. 3B). Increasing DMAb-V2L2 delivery (two plasmids, 200 ug total DNA) in combination with 10 mg/kg DepoMedrol extended DMAb expression in vivo to Day 25 (FIG. 3C).

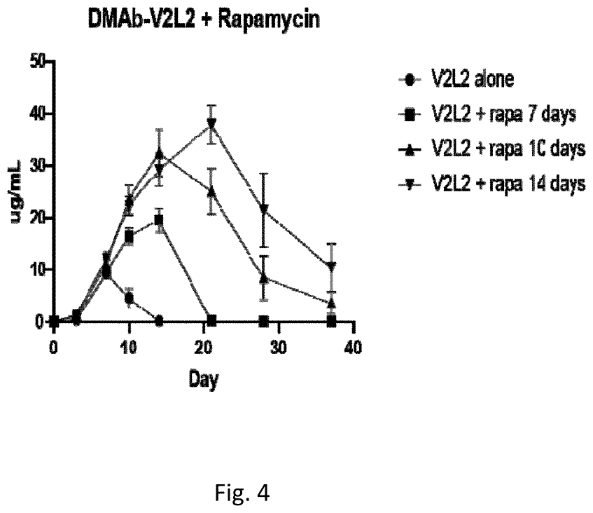

The mTOR pathway plays a critical role in intracellular signaling, including interleukin 2-mediated T cell proliferation and mTOR inhibitors are commonly administered to prevent rejection during solid organ transplantation. DMAb-V2L2 (two plasmids, 50 ug total DNA) was delivered alone or in combination with orally delivered rapamycin (0.5 mg/kg) daily for 7, 10, or 14 days. Co-delivery of rapamycin successfully increased the DMAb PK (FIG. 4). Continued administration of rapamycin enabled DMAb expression to continue to increase for 6 days beyond stopping treatment. A rapid decline in DMAb PK, characteristic of ADA development, was observed when rapamycin was stopped. Importantly, DMAb Cmax increased, providing additional data to support that PK is impaired by the development of ADA.

Blocking T-Cell Co-Stimulation with CTLA4-Ig

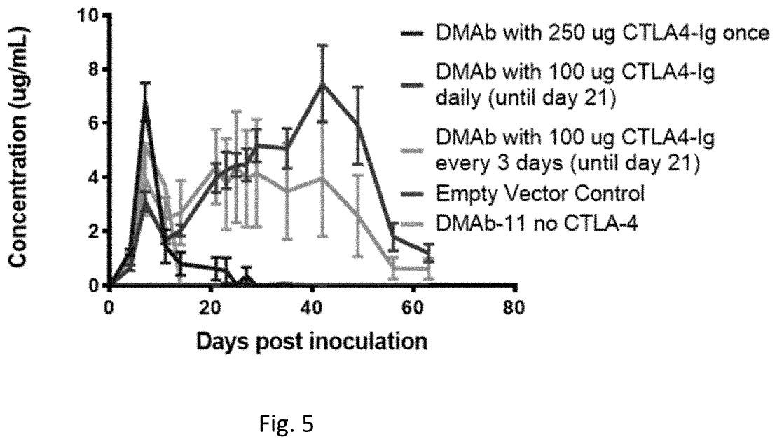

In addition to antigen presentation, co-stimulation sends important T cell activation signals to initiate adaptive immune responses. CD28 and TNF family co-stimulatory molecules both play important roles. CD28 interacts with CD80 (B7.1) and CD86 (B7.2) on APCs to initiate cell signaling pathways. In parallel, inhibitory molecule cytotoxic T lymphocyte-associate protein 4 (CTLA4/CD152) is an important member of the immune checkpoint pathway. DMAb-11 was delivered to BALB/c mice with and without intraperitoneal delivery of CTLA4-Ig at single 250 ug injection, 100 ug daily until day 21, 100 ug every 3 days until day 21 (FIG. 5A-FIG. 5F). A single CTLA4-Ig injection did not extend DMAb expression, however continued delivery daily or every 3 days resulted in extended DMAb expression. CTLA4-Ig delivery was stopped at day 21 and animals continued to express DMAb. However, did not have sustained expression as was previously observed with T cell depletion (Patel, A. et al., 2018, Cell Rep., 25(7):1982-1993).

Blocking Co-Stimulation with Anti-CD40L

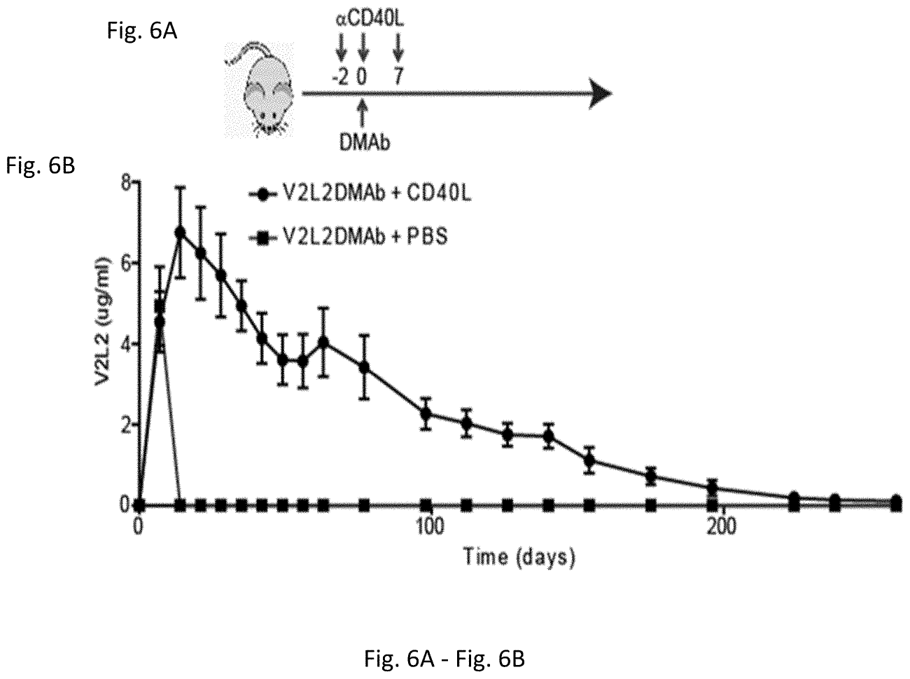

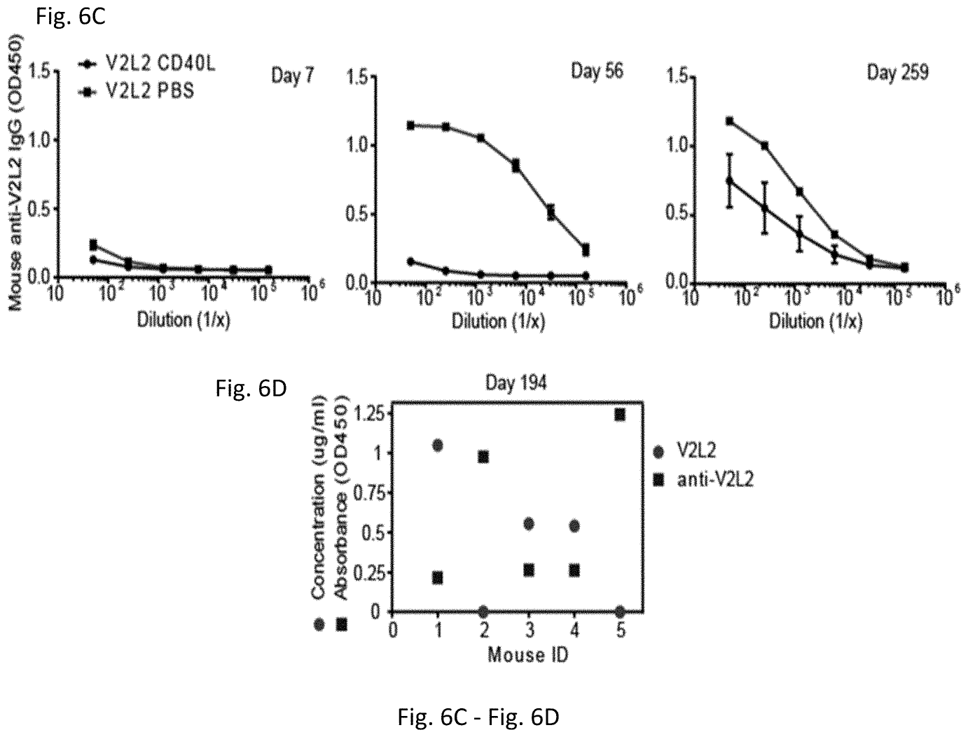

CD40L is a key signaling molecule whose role includes a major mechanism for TFH cells to activate B cells (Noelle R. J. et al., 1992, Proc. Natl. Acad. Sci. USA., 89(14):6550-6554). To investigate the effect of CD40L blockade in DMAb expression, the mouse CD40L blocking antibody MR1 was administered on days −2, 0 and 7 relative to the administration of the human IgG anti-Pseudomonas DMAb-V2L2 (FIG. 6A). Selective blockade of CD40L increased the time of circulating DMAb-V2L2 to over 200 days and increased the peak levels of expression (6.6 vs 10 ug/ml) (FIG. 6B). Mice that received anti-CD40L did not show ADA until after more than 170 days after DMAb expression (FIG. 6C). Interestingly, a significant negative correlation was found between the levels of ADA and V2L2 DMAb expression (r=−0.89, p=0.04) (FIG. 6D).

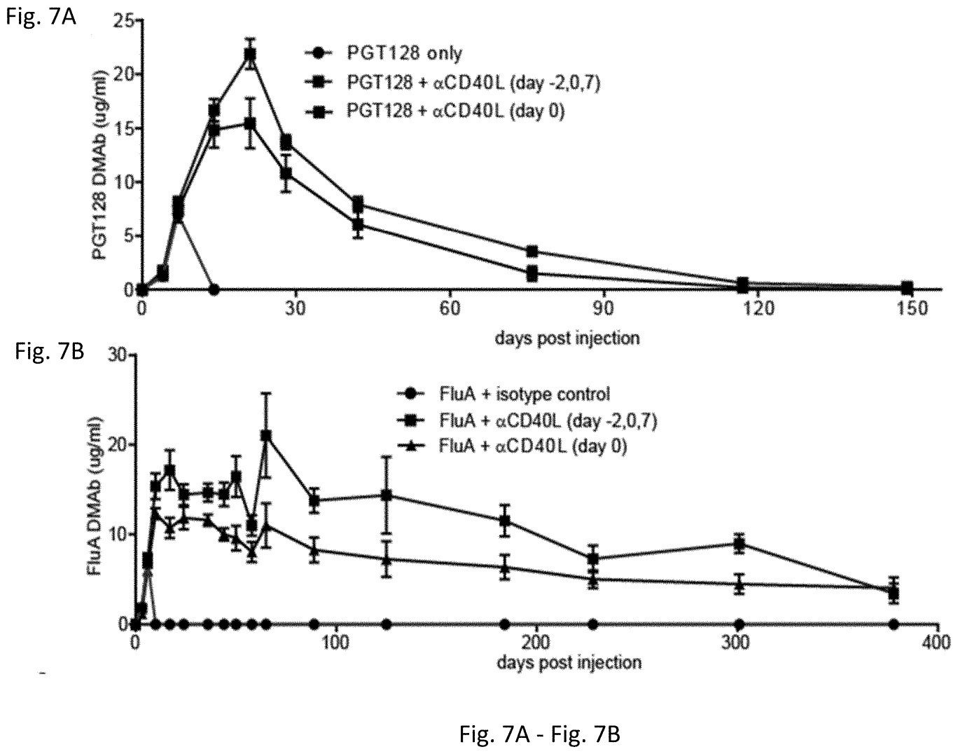

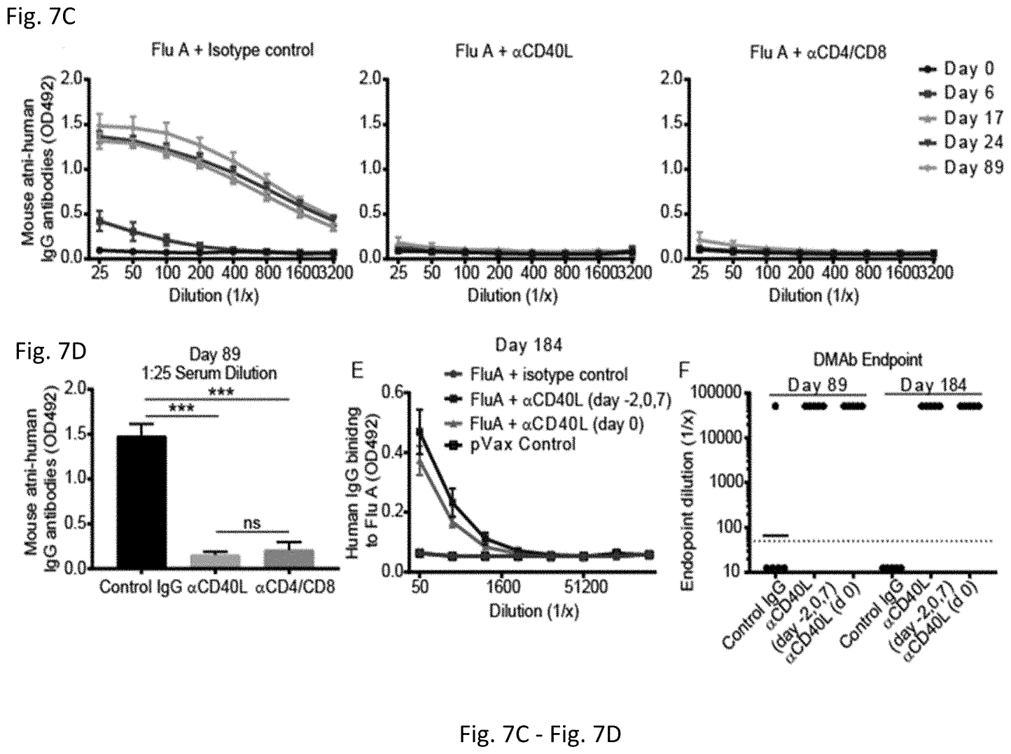

To confirm these results, the same anti-CD40L dosing with DMAb against a human HIV IgG (PGT128) and a human influenza A virus IgG (FY1-GL, here FluA) was repeated. In both cases, a marked increase in the time of expression of the human DMAbs in the mouse background was observed. PGT128 is an HIV broadly neutralizing antibody that could potentially be used for prophylaxis against HIV(Walker, L. M. et al., 2011, Nature, 477(7365):466-470). Expression of PGT128 DMAb increased from 14 to 150 days, reaching higher peak levels (8.6 vs 25 ug/m1) (FIG. 7A). This longer expression of circulating DMAb was similar to the levels obtained following T cell depletion. Similarly, longer expression (10 days vs >378 days) and a higher expression peak (8.6 vs 37.4 ug/ml) of FluA DMAb in anti-CD40L-treated versus untreated immunocompetent mice was found (FIG. 7B). This antibody is a fully human neutralizing antibody that reacts with all influenza A hemagglutinin subtypes and a closely-related iteration, MEDI8852, is being tested as therapy for influenza A (Kallewaard N. L. et al., 2016, Cell, 166(3):596-608). As with V2L2, significantly lower ADA was found, which was comparable to that induced in T cell depleted mice (FIG. 7C).

To determine that the use of CD40L blockade for increasing DMAb levels and circulating time did not interfere with the DMAb functionality, the ability of FluA DMAb to bind to its target, an influenza HA was measured. Sera from different timepoints was able to successfully bind to recombinant HA1 from Flu HA3 measured by a binding ELISA (FIG. 5E and FIG. 5F). This supports that the mAbs launched from the DMAb platform are functional for over long periods of time.

Single-Dose CD40L Blockade Prolongs DMAb similarly to 3 Doses

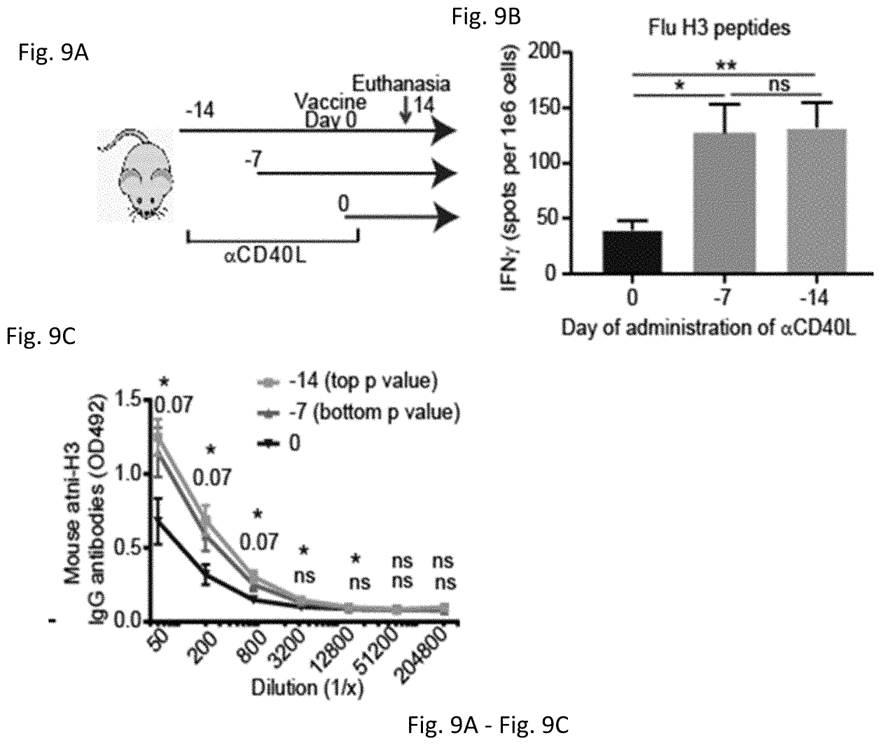

Next, a minimal treatment regime was investigated for its impact on in vivo expression of the DMAb, the effect of a single injection of the biologic anti-CD40L was examined at the same time of DMAb administration. A single injection of CD40L blocking antibody at the time of DMAb injection resulted in a similar extension of functional circulating antibody as the longer protocol of 3 injections (FIG. 7A, FIG. 7B, FIG. 7E, FIG. 7F). One dose of CD40L blockade at the time of DMAb administration significantly protected in vivo expression of the DMAb, suggesting blocking the initial event during DMAb expression is critical for modulation of ADA and resulting in significant effects for expression of xenogeneic IgG. To evaluate the effect of a single dose of CD40L blockade on cellular and humoral immune system and the time it would take to recover immune priming efficacy cohorts of mice with CD40L blockade were treated and administered a Flu H3 DNA vaccine at the same time, one or two weeks later (FIG. 9A). Vaccination 1 or 2 weeks after CD40L blockade resulted in significantly higher T cell and antibody responses against H3 Flu HA and viruses (FIG. 9B). suggesting temporal limitation of immune priming with this procedure.

The CD40-CD40L pathway is vital in the immune cell cross-activation. CD40 is expressed in B cells, dendritic cells, monocytes, platelets and macrophages and non-hematopoietic cells such as fibroblasts, epithelial and endothelial cells (Banchereau, J. et al., 1995, Adv. Exp. Med. Biol., 378:79-83). CD40 activation in B cells is necessary for the formation of the germinal center, immunoglobulin isotype switching, somatic hypermutations and formation of long-lived plasma cells and memory B cells (Van Kooten, C. et al., 1997, Curr. Opin. Immunol., 9(3):330-337). Therefore, preventing the CD40-CD40L mediated activation can be used to prevent the development of humoral adaptive immune responses against new antigens.

Based on these properties it was sought to examine the utility of acute CD40L blockade as a clinically available strategy to enhance therapy with DNA encoded monoclonal antibodies. It was demonstrated in murine models that blockade of CD40L achieves higher and longer levels of circulating xenogeneic IgG DMAbs in studies of three different antibodies. Furthermore, this clinically accessible intervention resulted in similar levels of expression as the absence of B or T cells.

DMAbs are a very attractive way to deliver monoclonal antibodies that is currently being tested in first-in-human clinical trials (NCT03831503). The DMAb platform enhances the availability of antibody treatment as DNA is more stable than proteins, and because it generates a temporary self-sustaining source of molecules encoding in this case antibodies, that can potentially last for months without the need of frequent re-administration. However, therapeutic antibodies are often isolated from heterologous non-human species and are frequently immunogenic in the new host. This immunogenicity eventually decreases the effectiveness of antibody therapy through human anti-antibody responses (Kuus-Reichel, K. et al., 1994, Clin. Diagn. Lab. Immunol., 1(4):365-372). Similarly, expression of human antibodies in mouse elicit an immune response that results in decreased antibody effectiveness, and in the DMAb model results in short-lived levels of antibodies in serum. The use of human mAbs in humans can also result immunogenic in clinical practice, leading to lower therapeutic efficacy due to ADA (Bartelds, G. M. et al., 2011, JAMA, 305(14):1460-1468; Harding, F. A. et al., 2010, MAbs, 2(3):256-265). Therefore, it could also be expected cases of human in human DMAbs to result in similar immunogenicity. CD40L blockade should be a good strategy to prevent ADA formation and extend circulating levels of immunogenic DMAbs in the clinic.

The immunomodulatory effect of CD40L blockade is effective in preventing the initiation of humoral immune responses irrespective of the Fc portion of the blocking antibody (Ferrant, J. L. et al., 2004, Int Immunol., 16(11):1583-1594). Due to the acute administration, the blockade of new humoral responses should in theory occur for a few weeks due to the half-life of the anti-CD40L antibody. Accordingly, using a model of vaccination, it was found that initiation of cellular and humoral responses significantly improved by one week after the administration of the anti-CD40L antibody, which was enough to provide over 4 months of DMAb expression.

Blockade of the CD40L-CD40 axis has undergone significant clinical development with different antibodies for the treatment of autoimmune diseases and transplantation (Zhang, T. et al., 2015, Immunotherapy, 7(8):899-911; Ford, M. L. et al., 2014, Nat. Rev. Nephrol., 10(1):14-24). However, thromboembolic events were observed associated with this treatment after repeating dose studies in a small percentage of patients (Boumpas, D. T. et al., 2003, Arthritis Rheum., 48(3):719-727). Recently, it has been determined that the reason for this event was due to platelet activation related to the IgG1 isotype of the anti-CD40L and newer versions of these molecules lacking Fc effector functions being developed for reintroduction into the clinical use (Xie, J. H. et al., 2014, J. Immunol., 192(9):4083-4092; Robles-Carrillo, L. et al., 2010, J. Immunol., 185(3):1577-1583). Acute blockade of CD40-CD40L axis could be used clinically to extend the peak level and time of expression of DMAbs. In the example of the FluA DMAb, this simple therapy allowed more than a year-long expression and virus binding activity from a single dose of DMAb in combination with CD40L blockade. This would be comparable to multiple injections of a biologic at much higher doses of material. In the case of HIV, even in the unlikely event of the antibody resulting as immunogenic in human as human PGT128 in mouse, treated individuals could obtain prophylaxis with a single injection every 3 months compared to a daily treatment as it is done today (Desai, M. et al., 2017, BMJ, 359). A single DMAb administration with CD40L blockade would facilitate therapeutic compliance and reduce cost in the delivery of important and effective treatments. In conclusion, acute blockade of CD40L signaling results in higher levels and long-term expression of DMAbs. This is a clinically relevant intervention that could expand the possibilities of clinical success for this novel strategy for delivering antibodies.

Sequences

DMAb-V2L2 HC
SEQ ID NO: 1 - nucleotide sequence
ATGGACTGGACCTGGAGAATCCTGTTCCTGGTGGCAGCAGCAACCGGAA
CACACGCAGAGGTGCAGCTGCTGGAGAGCGGCGGCGGCCTGGTGCAGCC
TGGCGGCAGCCTGAGGCTGTCCTGCGCAGCATCTGGCTTCACCTTTAGC
TCCTATGCAATGAACTGGGTGCGCCAGGCACCAGGCAAGGGACTGGAGT
GGGTGTCTGCCATCACAATGAGCGGCATCACCGCCTACTATACAGACGA
TGTGAAGGGCAGGTTTACCATCAGCAGAGACAACTCCAAGAATACACTG
TACCTGCAGATGAATAGCCTGAGAGCCGAGGATACCGCCGTGTACTATT
GCGCCAAGGAGGAGTTCCTGCCCGGCACACACTACTATTACGGAATGGA
CGTGTGGGGACAGGGAACCACAGTGACCGTGTCTAGCGCCTCCACAAAG
GGACCTAGCGTGTTCCCACTGGCACCCTCCTCTAAGTCCACCTCTGGCG
GCACAGCCGCCCTGGGCTGTCTGGTGAAGGATTATTTCCCAGAGCCCGT
GACCGTGTCTTGGAACAGCGGCGCCCTGACCTCTGGAGTGCACACATTT
CCAGCCGTGCTGCAGAGCTCCGGCCTGTATAGCCTGTCTAGCGTGGTGA
CCGTGCCCTCCTCTAGCCTGGGCACCCAGACATACATCTGCAACGTGAA
TCACAAGCCATCTAATACAAAGGTGGACAAGAAGGTGGAGCCCAAGAGC
TGTGATAAGACCCACACATGCCCTCCCTGTCCTGCACCAGAGCTGCTGG
GCGGCCCATCCGTGTTCCTGTTTCCACCCAAGCCTAAGGACACCCTGAT
GATCTCCCGGACCCCAGAGGTGACATGCGTGGTGGTGGACGTGTCTCAC
GAGGACCCCGAGGTGAAGTTCAACTGGTACGTGGATGGCGTGGAGGTGC
ACAATGCCAAGACCAAGCCACGGGAGGAGCAGTATAACAGCACCTACCG
CGTGGTGTCCGTGCTGACAGTGCTGCACCAGGACTGGCTGAACGGCAAG
GAGTACAAGTGCAAGGTGAGCAATAAGGCCCTGCCCGCCCCTATCGAGA
AGACCATCTCCAAGGCCAAGGGCCAGCCTAGGGAGCCACAGGTGTATAC
ACTGCCTCCAAGCAGAGACGAGCTGACCAAGAACCAGGTGTCCCTGACA
TGTCTGGTGAAGGGCTTCTACCCTTCCGATATCGCCGTGGAGTGGGAGT
CTAATGGCCAGCCAGAGAACAATTATAAGACCACACCCCCTGTGCTGGA
CTCCGATGGCTCTTTCTTTCTGTACTCTAAGCTGACCGTGGATAAGAGC
CGCTGGCAGCAGGGCAACGTGTTTAGCTGTTCCGTGATGCACGAGGCCC
TGCACAATCACTACACACAGAAGTCTCTGAGCCTGTCCCCTGGCAAGTG
ATAA
SEQ ID NO:2 - amino acid sequence
MDWTWRILFLVAAATGTHAEVQLLESGGGLVQPGGSLRLSCAASGFTFS
SYAMNWVRQAPGKGLEWVSAITMSGITAYYTDDVKGRFTISRDNSKNTL
YLQMNSLRAEDTAVYYCAKEEFLPGTHYYYGMDVWGQGTTVTVSSASTK
GPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKS
RWQQGNVFSCSVMHEALHNHYTQKSLSLSPGK
DMAb-V2L2 LC
SEQ ID NO:3 - nucleotide sequence
ATGGTGCTGCAGACACAGGTGTTCATCAGCCTGCTGCTGTGGATCTCCG
GAGCATACGGAGCAATCCAGATGACCCAGTCCCCAAGCTCCCTGAGCGC
CTCCGTGGGCGACAGGGTGACCATCACATGCAGAGCCTCTCAGGGCATC
CGGAACGATCTGGGCTGGTACCAGCAGAAGCCAGGCAAGGCCCCCAAGC
TGCTGATCTATTCTGCCAGCACCCTGCAGTCTGGAGTGCCCAGCCGGTT
CTCCGGCTCTGGCAGCGGAACAGACTTTACCCTGACAATCTCTAGCCTG
CAGCCTGAGGACTTCGCCACCTACTATTGCCTGCAGGATTACAATTATC
CATGGACCTTTGGCCAGGGCACAAAGGTGGAGATCAAGCGCACAGTGGC
CGCCCCCAGCGTGTTCATCTTTCCCCCTAGCGACGAGCAGCTGAAGTCC
GGCACCGCCTCTGTGGTGTGCCTGCTGAACAATTTCTACCCTAGGGAGG
CCAAGGTGCAGTGGAAGGTGGATAACGCCCTGCAGAGCGGCAATTCCCA
GGAGTCTGTGACCGAGCAGGACAGCAAGGATTCCACATATTCCCTGTCT
AACACCCTGACACTGAGCAAGGCCGATTACGAGAAGCACAAGGTGTATG
CATGCGAGGTGACCCACCAGGGACTGTCCTCTCCCGTGACAAAGTCCTT
TAATAGGGGCGAGTGTTGATAA
SEQ ID NO:4 - amino acid sequence
MVLQTQVFISLLLWISGAYGAIQMTQSPSSLSASVGDRVTITCRASQGI
RNDLGWYQQKPGKAPKLLIYSASTLQSGVPSRFSGSGSGTDFTLTISSL
QPEDFATYYCLQDYNYPWTFGQGTKVEIKRTVAAPSVFIFPPSDEQLKS
GTASVVCLLNNFYPREAKVQWKVDNALQSGNSQESVTEQDSKDSTYSLS
NTLTLSKADYEKHKVYACEVTHQGLSSPVTKSFNRGEC
DMAb-11-HC
SEQ ID NO:5 - nucleotide sequence
ATGGACTGGACCTGGAGAATCCTGTTCCTGGTGGCAGCAGCAACCGGAA
CACACGCAGAGGTGCAGCTGGTGGAGAGCGGCGGCGGCCTGATCCAGCC
AGGCGGCAGCCTGAGGCTGTCCTGCGCAGCATCTGGATTTGCCGTGAGG
AGCAACTACCTGTCCTGGGTGAGACAGGCACCAGGCAAGGGACTGGAGT
GGGTGTCTCTGATCTACAGCGGCGGCCTGACCGCATATGCAGACAGCGT
GGAGGGCAGGTTCACCATCTCCAGAGATAACTCTAAGAATACACTGTAT
CTGCAGATGAATTCCCTGCGGGTGGAGGACACCGCCCTGTACTATTGCG
CCCGCGTGGCCAGCTCCGCCGGCACATTCTACTATGGCATGGACGTGTG
GGGCCAGGGCACCACAGTGACCGTGTCTAGCGC
SEQ ID NO:6 - amino acid sequence
MDWTWRILFLVAAATGTHAEVQLVESGGGLIQPGGSLRLSCAASGFAVR
SNYLSWVRQAPGKGLEWVSLIYSGGLTAYADSVEGRFTISRDNSKNTLY
LQMNSLRVEDTALYYCARVASSAGTFYYGMDVWGQGTTVTVSS
DMAb11-LC
SEQ ID NO:7 - nucleotide sequence
ATGGTGCTGCAGACCCAGGTGTTTATCTCTCTGCTGCTGTGGATCAGCG
GCGCCTACGGCGATATCGTGATGACCCAGTCCCCTCGCTCCCTGTCTGT
GACACCTGGCGAGCCAGCCAGCATCTCCTGTCGGTCCTCTCAGTCTCTG
CTGCACCGCAACGGCTACAATTATCTGGACTGGTACCTGCAGAAGCCCG
GCCAGTCCCCTCAGCTGCTGATCTATCTGGGCAGCAACAGGGCATCCGG
AGTGCCAGACCGCTTCTCTGGCAGCGGCTCCGGAACCGACTTCACCCTG
AAGATCAGCAGGGTGGAGGCCGAGGATGTGGGCGTGTACTATTGCATGC
AGGCCCTGCAGACCCCCTCCTGGACATTCGGCCAGGGCACCAAGGTGGA
GATCAAG
SEQ ID NO:8 - amino acid sequence
MVLQTQVFISLLLWISGAYGDIVMTQSPRSLSVTPGEPASISCRSSQSL
LHRNGYNYLDWYLQKPGQSPQLLIYLGSNRASGVPDRFSGSGSGTDFTL
KISRVEAEDVGVYYCMQALQTPSWTFGQGTKVEIK
Flu B
SEQ ID NO:9 - nucleotide sequence
atggactggacttggaggattctgtttctggtggccgccgcaactggca
ctcatgccgaggtgcagctggtggaatcagggggaggactggtgaagcc
tggcggatcactgcgactgagctgcgcagcttccggactgaccttcctg
aacgcttggatgagctgggtgcgacaggcaccagggaaaggcctggaat
gggtcgggcgcatcaagagcaatacagacggcggaaccacagattacgc
agcccccgtgaaaggcaggttcaccatttctcgggacgatagtaagaac
acactgtatctgcagatgagctccctgaaaaccgaggacacagccgtgt
actattgcactaccgatggcccctacagcgacgatttccgctccggata
tgctgcacggtaccgctattttgggatggacgtgtggggacaggggaca
actgtcacagtgtctagtgcatctactaagggacctagcgtgttcccac
tggccccctcaagcaaateaactagcggagggaccgccgctctgggatg
tctggtgaaggattacttccccgagcctgtcaccgtgagctggaactcc
ggggccctgacctccggagtgcacacatttcctgctgtcctgcagtcct
ctgggctgtactctctgagttcagtggtcacagtgccaagctcctctct
gggcactcagacctatatctgcaacgtgaatcacaaacctagcaatact
aaggtcgacaagaaagtggaaccaaaaagctgtgataagacacatactt
gccctccctgtccagctccagagctgctgggcggaccatccgtgttcct
gtttccacccaagcccaaagacaccctgatgatttcccggacaccagaa
gtgacttgcgtggtcgtggacgtgagccacgaggaccccgaagtgaagt
tcaactggtacgtggatggcgtcgaggtgcataatgccaagacaaaacc
cagggaggaacagtacaactcaacttatagagtcgtgagcgtcctgacc
gtgctgcaccaggactggctgaacggcaaggagtataagtgcaaagtga
gcaacaaggccctgcctgctccaatcgagaagactattagcaaggctaa
aggacagcctcgggaaccacaggtgtacaccctgcctccatcccgcgac
gagctgaccaaaaaccaggtgtctctgacatgtctggtcaagggcttct
atccctctgatatcgccgtggagtgggaaagtaatggacagcctgaaaa
caattacaagaccacaccccctgtgctggactctgatggcagtttcttt
ctgtatagtaaactgaccgtggacaagtcaagatggcagcagggaaacg
tgttttcctgctctgtcatgcatgaggccctgcacaatcattacaccca
gaagagtctgtcactgagcccaggaaaacgagggaggaagaggagatcc
ggctctggagccacaaacttctccctgctgaagcaggctggagacgtgg
aggaaaatcccgggcctatggtgctgcagacccaggtctttatctccct
gctgctgtggatttctggcgcttacggagatatccagatgacacagtct
cccagttcagtcagtgcatcagtgggcgaccgcgtcaccatcacatgtc
gagcatcacaggatattagcacctggctggcctggtaccagcagaagcc
cggaaaagctcctaagctgctgatctatgcagccagctccctgcagtcc
ggagtgccctctaggttcagcgggtccggctctggaacagactttactc
tgaccatttctagtctgcagcctgaggatttcgcaacttactattgcca
gcaggccaacagcttcccacccacttttgggcagggcaccaaactggaa
atcaagactgtggctgcacctagcgtcttcatttttcctccatccgacg
agcagctgaagagtggcaccgcctcagtggtgtgcctgctgaacaactt
ctacccaagagaagcaaaagtgcagtggaaggtcgataacgccctgcag
tcaggcaatagccaggagtccgtgacagaacaggactctaaggatagta
cttatagtctgtcaaatacactgactctgagcaaagctgactacgagaa
gcataaagtgtatgcatgcgaggtcactcaccagggactgtcttcaccc
gtcaccaaatctttcaatagaggagaatgctgataa
SEQ ID NO: 10 - amino acid sequence
MDWTWRILFLVAAATGTHAEVQLVESGGGLVKPGGSLRLSCAASGLTFL
NAWMSWVRQAPGKGLEWVGRIKSNTDGGTTDYAAPVKGRFTISRDDSKN
TLYLQMSSLKTEDTAVYYCTTDGPYSDDFRSGYAARYRYFGMDVWGQGT
TVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNT
KVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFF
LYSKLTVDKSRWQQGNVFSCSVMHEALHNHYTQKSLSLSPGKRGRKRrS
GSGATNFSLLKQAGDVEENPGPMVLQTQVFISLLLWISGAYGDIQMTQS
PSSVSASVGDRVTITCRASQDISTWLAWYQQKPGKAPKLLIYAASSLQS
GVPSRFSGSGSGTDFTLTISSLQPEDFATYYCQQANSFPPTFGQGTKLE
IKTVAAPSVFIFPPSDEQLKSGTASVVCLLNNFYPREAKVQWKVDNALQ
SGNSQESVTEQDSKDSTYSLSNTLTLSKADYEKHKVYACEVTHQGLSSP
VTKSFNRGEC
PGT128_HC
SEQ ID NO: 11 - nucleotide sequence
ATGGACTGGACCTGGAGAATCCTGTTCCTGGTGGCAGCAGCAACAGGAA
CCCACGCACAGCCACAGCTGCAGGAGTCCGGACCCACCCTGGTGGAGGC
CTCCGAGACACTGTCTCTGACCTGCGCCGTGAGCGGCGATTCCACAGCA
GCCTGTAACTCCTTCTGGGGATGGGTGCGCCAGCCCCCTGGCAAGGGCC
TGGAGTGGGTGGGCTCTCTGAGCCACTGCGCCAGCTACTGGAACAGGGG
CTGGACCTATCACAATCCCTCTCTGAAGAGCAGACTGACCCTGGCCCTG
GACACACCTAAGAACCTGGTGTTCCTGAAGCTGAATAGCGTGACCGCCG
CCGATACAGCCACCTACTATTGTGCCAGGTTTGGCGGCGAGGTGCTGAG
ATACACAGACTGGCCAAAGCCAGCATGGGTGGATCTGTGGGGAAGGGGC
ACACTGGTGACCGTGAGCTCCGCCTCCACCAAGGGACCAAGCGTGTTCC
CACTGGCACCTTCTAGCAAGTCCACATCTGGCGGCACCGCCGCCCTGGG
ATGCCTGGTGAAGGACTACTTCCCTGAGCCAGTGACAGTGTCCTGGAAC
TCTGGCGCCCTGACCTCTGGCGTGCACACATTTCCCGCCGTGCTGCAGT
CCTCTGGCCTGTACAGCCTGAGCTCCGTGGTGACCGTGCCTTCTAGCTC
CCTGGGCACACAGACCTATATCTGCAACGTGAATCACAAGCCTAGCAAT
ACAAAGGTGGACAAGAAGGTGGAGCCAAAGTCCTGTGATAAGACACACA
CCTGCCCACCCTGTCCAGCACCTGAGCTGCTGGGCGGCCCTTCCGTGTT
CCTGTTTCCTCCAAAGCCAAAGGACACCCTGATGATCTCCCGGACACCT
GAGGTGACCTGCGTGGTGGTGGACGTGTCTCACGAGGACCCCGAGGTGA
AGTTTAACTGGTACGTGGATGGCGTGGAGGTGCACAATGCCAAGACCAA
GCCCAGGGAGGAGCAGTACAACTCTACCTATAGAGTGGTGAGCGTGCTG
ACAGTGCTGCACCAGGATTGGCTGAACGGCAAGGAGTATAAGTGCAAGG
TGAGCAATAAGGCCCTGCCAGCCCCCATCGAGAAGACCATCTCCAAGGC
AAAGGGACAGCCACGGGAGCCACAGGTGTACACACTGCCCCCTTCCCGC
GACGAGCTGACCAAGAACCAGGTGTCTCTGACATGTCTGGTGAAGGGCT
TCTATCCAAGCGATATCGCCGTGGAGTGGGAGTCCAATGGCCAGCCCGA
GAACAATTACAAGACCACACCACCCGTGCTGGACAGCGATGGCTCCTTC
TTTCTGTATTCCAAGCTGACCGTGGACAAGTCTAGGTGGCAGCAGGGCA
ACGTGTTTAGCTGCTCCGTGATGCACGAGGCCCTGCACAATCACTACAC
CCAGAAGTCTCTGAGCCTGTCCCCTGGCAAGTGATAA
SEQ ID NO: 12 - amino acid sequence
MDWTWRILFLVAAATGTHAQPQLQESGPTLVEASETLSLTCAVSGDSTA
ACNSFWGWVRQPPGKGLEWVGSLSHCASYWNRGWTYHNPSLKSRLTLAL
DTPKNLVFLKLNSVTAADTATYYCARFGGEVLRYTDWPKPAWVDLWGRG
TLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSN
TKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSF
FLYSKLTVDKSRWQQGNVFSCSVMHEALHNHYTQKSLSLSPGK
PGT128-LC
SEQ ID NO: 13 - nucleotide sequence
ATGGCCTGGACCCCTCTGTTCCTGTTCCTGCTGACATGCTGTCCTGGCG
GCTCCAACTCTCAGAGCGCCCTGACCCAGCCTCCATCCGCCTCTGGCAG
CCCTGGACAGAGCATCACAATCTCCTGTACAGGCACCAGCAACAATTTC
GTGAGCTGGTACCAGCAGCACGCAGGCAAGGCACCAAAGCTGGTCATCT
ACGACGTGAACAAGCGGCCTTCCGGCGTGCCAGATCGCTTCTCCGGCTC
TAAGAGCGGCAATACAGCCTCTCTGACCGTGAGCGGCCTGCAGACCGAC
GATGAGGCCGTGTACTATTGCGGCAGCCTGGTGGGCAACTGGGACGTGA
TCTTCGGCGGCGGAACAAAGCTGACCGTGCTGGGACAGCCAAAGGCAGC
ACCTTCCGTGACCCTGTTTCCCCCTTCTAGCGAGGAGCTGCAGGCCAAT
AAGGCCACCCTGGTGTGCCTGATCAGCGACTTCTACCCTGGAGCAGTGA
CAGTGGCATGGAAGGCCGATTCCTCTCCAGTGAAGGCCGGCGTGGAGAC
CACAACCCCCTCTAAGCAGAGCAACAATAAGTACGCCGCCAGCTCCTAT
CTGTCTCTGACCCCAGAGCAGTGGAAGAGCCACAAGTCCTATTCTTGCC
AGGTGACACACGAGGGCTCTACAGTGGAGAAGACCGTGGCCCCCACAGA
GTGTAGCTGATAA
SEQ ID NO: 14 - amino acid sequence
MAWTPLFLFLLTCCPGGSNSQSALTQPPSASGSPGQSITISCTGTSNNF
VSWYQQHAGKAPKLVIYDVNKRPSGVPDRFSGSKSGNTASLTVSGLQTD
DEAVYYCGSLVGNWDVIFGGGTKLTVLGQPKAAPSVTLFPPSSEELQAN
KATLVCLISDFYPGAVTVAWKADSSPVKAGVETTTPSKQSNNKYAASSY
LSLTPEQWKSHKSYSCQVTHEGSTVEKTVAPTECS

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variation.

1. A composition comprising an inhibitor of B cell maturation or function and further comprising one or more nucleic acid molecules encoding one or more synthetic antibodies or fragments thereof.

2. The composition of claim 1, wherein the inhibitor of B cell maturation or function is selected from the group consisting of an inhibitor of CD40L, CD20, CD22, VLA-4, BAFF or APRIL.

3. The composition of claim 1, wherein the inhibitor of B cell maturation or function is selected from the group consisting of intravenous gamma globuli, interferon-β, DC2219, MR1, rituximab, Ocrelizumab, Epratuzumab, Atacicept, natalizumab and Belimumab.

4. The composition of claim 1, wherein the nucleic acid molecules encodes a DNA encoded monoclonal antibody (DMAb), an ScFv DMAb, a DNA encoded bispecific T cell engager, a bispecific antibody, a chimeric antibody, or a functional antibody fragment.

5. The composition of any one of claims 1-4, wherein the one or more nucleic acid molecules are engineered to be in an expression vector.

6. The composition of claim 1, further comprising a checkpoint inhibitor, or nucleic acid molecule encoding the same.

7. The composition of claim 1, further comprising a pharmaceutically acceptable excipient.

8. A method of treating a disease in a subject, the method comprising administering to the subject any composition of claims 1-7.

9. The method of claim 8, wherein the disease is selected from the group consisting of a bacterial infection, a viral infection, a fungal infection, a disease or disorder associated with a parasite, and cancer.

10. A method of extending the duration of circulation of a synthetic antibody, the method comprising administering to a subject in need thereof:
a) an inhibitor of B cell maturation or function, and
b) a composition comprising one or more nucleic acid molecule encoding a synthetic antibody.

11. The method of claim 10, wherein the inhibitor of B cell maturation or function is selected from the group consisting of an inhibitor of CD40L, CD20, CD22, VLA-4, BAFF or APRIL.

12. The method of claim 10, wherein the inhibitor of B cell maturation or function is selected from the group consisting of intravenous gamma globuli, interferon-β, DC2219, MR1, rituximab, Ocrelizumab, Epratuzumab, Atacicept, natalizumab and Belimumab.

13. The method of claim 10, wherein the nucleic acid molecules encodes a DNA encoded monoclonal antibody (DMAb), an ScFv DMAb, a DNA encoded bispecific T cell engager, a bispecific antibody, a chimeric antibody, or a functional antibody fragment.

14. The method of claim 10, wherein the one or more nucleic acid molecules are engineered to be in an expression vector.

15. The method of claim 10, wherein administering the composition comprises an electroporating step.

16. The method of claim 10, further comprising a step of administering to the subject a composition comprising an antigen.

17. A method of treating or preventing a disease or disorder, the method comprising administering to a subject in need thereof:
a) an inhibitor of B cell maturation or function, and
b) a composition comprising one or more nucleic acid molecule encoding a synthetic antibody.

18. The method of claim 17, wherein the inhibitor of B cell maturation or function is selected from the group consisting of an inhibitor of CD40L, CD20, CD22, VLA-4, BAFF or APRIL.

19. The method of claim 17, wherein the inhibitor of B cell maturation or function is selected from the group consisting of intravenous gamma globuli, interferon-β, DC2219, MR1, rituximab, Ocrelizumab, Epratuzumab, Atacicept, natalizumab and Belimumab.

20. The method of claim 17, wherein the nucleic acid molecules encodes a DNA encoded monoclonal antibody (DMAb), an ScFv DMAb, a DNA encoded bispecific T cell engager, a bispecific antibody, a chimeric antibody, or a functional antibody fragment.

21. The method of claim 17, wherein the one or more nucleic acid molecules are engineered to be in an expression vector.

22. The method of claim 17, wherein administering the composition comprises an electroporating step.

23. The method of claim 17, further comprising a step of administering to the subject a composition comprising an antigen.

24. The method of claim 17, wherein the disease is selected from the group consisting of a bacterial infection, a viral infection, a fungal infection, a disease or disorder associated with a parasite, and cancer.
